(19) United States
(12) Patent Application Publication
(10) Pub. No.: US 2014/0350222 A1
(43) Pub. Date: Nov. 27, 2014

(54) Microalgal Extraction

(71) Applicants: FLINDERS UNIVERSITY OF SOUTH AUSTRALIA, Bedford Park (AU); MINISTER FOR AGRICULTURE , FOOD AND FISHERIES, South Australia (AU)

(72) Inventors: Wei Zhang, St Georges (AU); Peng Su, Oaklands Park (AU)

(21) Appl. No.: 14/352,346

(22) PCT Filed: Oct. 19, 2012
(86) PCT No.: PCT/AU2012/001279

§ 371 Date: Apr. 17, 2014

(30) Foreign Application Priority Data
Oct. 20, 2011 (AU) 2011904343

(51) Int. Cl. C07K 1/14; C07H 1/08; C11B 1/10

(52) U.S. Cl. CPC C07K 1/145; C11B 1/10; C07H 1/08
USPC 530/350; 554/175; 536/127; 530/427

(57) ABSTRACT

Disclosed herein is a process for extracting lipid containing products from microalgal biomass, the process comprising: (i) treating an aqueous mixture comprising microalgal biomass with microwave radiation and (ii) recovering lipid containing products from the treated microalgal biomass.

### PRIORITY DOCUMENTS

The present application claims priority from Australian Provisional Patent Application No. 2011904343 entitled “MICROALGAL EXTRACTION” filed on 20 Oct. 2011 the contents of which are hereby incorporated by reference in their entirety.

### TECHNICAL FIELD

The present invention relates to processes for extracting materials from microalgal biomasses.

### BACKGROUND

Fossil fuels are not a long term prospect as a transport fuel. This, combined with the negative environmental impacts of burning fossil fuels, has led to the search for renewable energy sources that can provide an alternative to non-renewable fossil fuels. Biofuels are one such renewable energy source. Biofuels are fuels produced from renewable organic sources or feedstocks. The term generally refers to fuels for transportation and includes ethanol and biodiesel. To date, the main source of oil for biodiesel production has been food crops. However, there are also negative environmental impacts associated with the use of food crops and they are unlikely to be able to supply enough fuel for transportation. Biomass such as grasses, residue from grain crops, woodland products or waste, and the like may be used to produce biofuels, but they are generally used for the production of bioethanol. However, bioethanol is not optimum as a fuel source.

The use of algae as a renewal organic feedstock that can be used to produce oils suitable for biodiesel production has several advantages when compared to the use of other renewable organic sources. Some algae produce considerable amounts of oils or lipids. For example, some algae contain up to 80% oil by weight and, as such, they can provide an abundant source of oils for the production of biodiesel. Furthermore, algae are rapidly growing and can produce 10 to 100 times as much mass as terrestrial plants in a year and they will grow in a wide range of environmental conditions.

The oil extracted from algae is a mixture of triglycerides and various lipophilic pigments. The oil can be used as a fuel, either directly or indirectly by conversion to biodiesel via transesterification.

Unfortunately, the production of oil from algae has proven to be a difficult and/or expensive process on a commercial scale. Typically, the microalgal biomass is pumped from the growing pond, tank or vessel to a centrifuge or decanter where the volume of the slurry is reduced to about 80% of the starting volume. A further drying step is then typically carried out prior to the oil extraction step. The drying step requires time and significant energy input and therefore adds to the overall costs of the process.

Production of oil from algae requires extraction of the oil from the biomass and, preferably, purification of the lipid fractions from other organic contaminants. In some processes, the oil is extracted by solvent extraction using an organic solvent. For example, U.S. Pat. No. 6,166,231 describes a two-phase solvent extraction of oil from biomass. U.S. Pat. No. 5,458,897 discloses methods for extracting volatile oils from plant material, animals and soils by mixing them with a non-aqueous solvent and exposing the mixture to microwave radiation.

The drying and/or solvent extraction steps require time and add cost to the extraction process.

There is a need for processes for extracting oil and other products from algae that overcome one or more of the problems associated with prior art processes.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

### SUMMARY

The present invention provides a new process for the economically viable production of oil from microalgae. Specifically, the present invention arises from research into processes for extracting and fractionating commercial products from microalgal biomass and, in particular, our discovery that exposing wet microalgal biomass to microwave radiation leads to the release of oil products which can be recovered without the need for an intermediate drying step.

In one aspect, the present invention provides a process for extracting lipid containing products from microalgal biomass, the process comprising: (i) treating an aqueous mixture comprising microalgal biomass with microwave radiation and (ii) recovering lipid containing products from the treated microalgal biomass.

Advantageously, the microalgal biomass may be a “wet” biomass. The microalgal biomass may comprise up to 90% water. Extraction of lipid containing products from “wet” biomass means that there is no need to remove any of the water from the microalgal biomass and this leads to considerable efficiency in the extraction process as a separate drying step that is normally required is both time and energy intensive.

In some embodiments, the microalgal biomass comprises about 10% to about 90% by weight of water.

We have also found that carbohydrate materials can be selectively extracted from microalgal biomass by decreasing the pH of the aqueous mixture comprising the biomass. Thus, the present invention also provides a method for extracting lipid containing products and carbohydrate containing products from microalgal biomass, the process comprising: (i) providing an aqueous mixture containing the microalgal biomass; (ii) adjusting the pH of the aqueous mixture to pH<7; (iii) heating the aqueous mixture containing the microalgal biomass; and (iv) separating the lipid containing products and the carbohydrate containing products from the biomass.

Additionally, we have found that protein materials can be selectively extracted from microalgal biomass by increasing the pH of the aqueous mixture comprising the biomass. Thus, the present invention also provides a method for extracting lipid containing products and protein containing products from microalgal biomass, the process comprising: (i) providing an aqueous mixture containing the microalgal biomass; (ii) adjusting the pH of the aqueous mixture to pH>7; (iii) heating the aqueous mixture containing the microalgal biomass; and (iv) separating the lipid containing products and the protein containing products from the biomass.

The processes described herein may be performed sequentially. As such, the present invention provides a process for selectively extracting lipid containing products, carbohydrate containing products, and protein containing products from microalgal biomass. This is advantageous for several reasons. Firstly, the lipid containing products obtained are substantially free of carbohydrate materials and protein materials. Secondly, the carbohydrate materials and protein materials are value added products that provide an additional revenue stream.

In some embodiments, the method comprises a first extraction at pH<7 followed by one or more extractions at pH>7 followed by recovery of the lipid containing product. Thus, the present invention provides a process for extracting lipid containing products, carbohydrate containing products, and protein containing products from microalgal biomass, the process comprising:
(i) providing an initial aqueous mixture containing the microalgal biomass;
(ii) adjusting the pH of the initial aqueous mixture to pH<7;
(iii) heating the acidic initial aqueous mixture containing the microalgal biomass to provide a first treated mixture;
(iv) separating the solid and the liquid from the first treated mixture to provide a first solid and a carbohydrate containing liquid;
(v) combining the first solid with an aqueous mixture to form a second aqueous mixture containing microalgal biomass;
(vi) adjusting the pH of the second aqueous mixture to pH>7;
(vii) heating the alkaline second aqueous mixture containing the microalgal biomass to provide a second treated mixture;
(viii) separating the solid and the liquid from the second treated mixture to provide a second solid and a protein containing liquid;
(ix) optionally, repeating steps (v) to (viii) using the second solid to provide a third solid and a further protein containing liquid;
(x) combining the second solid or the third solid with an aqueous mixture to form a final aqueous mixture;
(xi) treating the final aqueous mixture with a solvent;
(xii) separating the solvent from the final aqueous mixture to provide a solvent containing lipid product.

In some embodiments, the pH of the mixture formed in step (ii) is in the range of from about 0.5 to about 2. In some specific embodiments, the pH of the mixture formed in step (ii) is about 1.

In some embodiments, the pH of the mixture formed in step (vi) is in the range of from about 11 to about 14. In some specific embodiments, the pH of the mixture formed in step (iv) is about 13.

The steps of heating the acidic or alkaline aqueous mixtures containing the microalgal biomass may be carried out using any suitable heat source. The heating step(s) may be carried out at atmospheric pressure or at above atmospheric pressure. In some embodiments, the steps of heating the acidic or alkaline aqueous mixtures containing the microalgal biomass are carried out by treating the mixtures with microwave radiation.

The solvent used in the step of treating the final aqueous mixture with a solvent may be any non-aqueous solvent.

In another aspect, the present invention provides a product produced by the process described herein.

### BRIEF DESCRIPTION OF THE ACCOMPANYING FIGURES

FIG. 1 is a plot showing a comparison of carbohydrate extraction yield by different microwave irradiation times under acidic and alkaline conditions.

FIG. 2 is a plot showing a comparison of protein extraction yield by different microwave irradiation times under acidic and alkaline condition.

FIG. 3 is a plot showing the carbohydrate extraction yield of six repeated microwave extractions under acidic conditions only.

FIG. 4 is a plot showing the protein extraction yield of six repeated microwave extractions under acidic conditions only.

FIG. 5 is a plot showing the carbohydrate extraction yield of six repeated microwave extractions under alkaline conditions only.

FIG. 6 is a plot showing the protein extraction yield of six repeated microwave extractions under alkaline conditions only.

FIG. 7 is a plot showing the carbohydrate extraction yield of six repeated microwave extractions under alkaline to acidic conditions.

FIG. 8 is a plot showing the protein extraction yield of six repeated microwave extractions under alkaline to acidic conditions.

FIG. 9 is a plot showing the carbohydrate extraction yield of six repeated microwave extractions under acidic to alkaline conditions.

FIG. 10 is a plot showing the protein extraction yield of six repeated microwave extractions under acidic to alkaline conditions.

FIG. 11 is a plot showing extracted carbohydrate recovery test by glucose spiking.

FIG. 12 is a plot showing extracted carbohydrate recovery test by BSA spiking.

FIG. 13 is a photograph of extracted microalgal lipid floating on the surface of the extraction buffer.

FIG. 14 is a plot showing a comparison of extracted carbohydrate yields of six repeated microwave extractions under acidic to alkaline conditions from 2.5 g biomass and 25 g biomass.

FIG. 15 is a plot showing a comparison of extracted carbohydrate yields of six repeated microwave extractions under acidic to alkaline conditions from 2.5 g biomass and 25 g biomass.

FIG. 16 is a plot showing the protein extraction efficiency of six repeated microwave extractions by the micro-lowry method.

FIG. 17 is a plot showing the protein extraction efficiency of the six repeated microwave extractions by the micro-lowry method.

FIG. 18 is a plot showing a comparison of the protein extraction yield between extraction processes with 1:10 biomass and buffer ratio and 1:5 biomass and buffer ratio.

FIG. 19 is a plot showing the protein extraction efficiency of the seven repeated microwave extractions using wet biomass.

FIG. 20 is a plot showing the carbohydrate extraction efficiency of the seven repeated microwave extractions using wet biomass.

FIG. 21 is a plot showing the lipid content analysis of wet, freeze-dried and oven-dried biomass.

FIG. 22 is a plot showing the chemical characterisation of algal biomass.

FIG. 23 is a flow chart showing the experimental design for optimising microwave extraction conditions of wet algal biomass.

FIG. 24 is a plot showing a comparison of ash-free protein extraction efficiency using wet algal biomass.

FIG. 25 is a plot showing a comparison of ash-free reducing sugar extraction efficiency using wet algal biomass.

FIG. 26 is a plot showing a comparison of ash-free carbohydrate extraction efficiency using wet algal biomass.

FIG. 27 is a plot showing a comparison of ash-free protein extraction efficiency with three microwave extractions.

FIG. 28 is a plot showing a comparison of ash-free carbohydrate extraction efficiency with three microwave extractions.

FIG. 29 is a plot showing a comparison of protein and carbohydrate recovery with three microwave extractions.

FIG. 30 is a plot showing a material mass balance of the microwave extraction process.

FIG. 31 is a plot showing lipid recovery and lost rate of the microwave extraction process.

FIG. 32 is a plot showing ash-free carbohydrate extraction efficiency with microwave extractions at different pH.

FIG. 33 is a plot showing ash-free protein extraction efficiency with microwave extractions at different pH.

FIG. 34 is a plot showing ash-free reducing sugar extraction efficiency with microwave extractions at different pH.

FIG. 35 is a plot showing total carbohydrate and protein recovery with microwave extractions at different pH.

FIG. 36 is a plot showing ash-free protein extraction efficiency using different heating methods.

FIG. 37 is a plot showing ash-free carbohydrate extraction efficiency using different heating methods.

FIG. 38 is a plot showing total carbohydrate, protein lipid and ash content.

FIG. 39 is a plot showing a comparison of ash-free protein extraction efficiency with different microwave extraction temperatures and times.

FIG. 40 is a plot showing a comparison of ash-free total carbohydrate extraction efficiency with different microwave extraction temperatures and times.

FIG. 41 is a plot showing a comparison of total protein and carbohydrate productivity based on power consumption of microwave extraction with different extraction temperatures and times.

FIG. 42 is a plot showing a comparison of total power consumption of different extraction conditions.

FIG. 43 is a plot showing a comparison of total protein and carbohydrate productivity based on power consumption with different extraction conditions at 25 mL sample scales.

FIG. 44 is a lot showing a comparison of total protein and carbohydrate productivity based on power consumption of microwave extraction with different sample scales.

### DETAILED DESCRIPTION

In a first aspect, the present invention provides a process for extracting lipid containing products from microalgal biomass, the process comprising: (i) treating an aqueous mixture comprising microalgal biomass with microwave radiation and (ii) recovering lipid containing products from the treated microalgal biomass.

As used herein, the terms “microalgae”, “microalgal” and related terms means any unicellular, photosynthetic microorganism. Microalgae are also referred to as phytoplankton, microphytes, or planktonic algae. Typical microalgae include green algae (Chlorophyta) and blue-green algae (Cyanophyta).

As used herein, the term “biomass” means the biological material from living or recently living organisms.

As used herein, the term “lipid” means any organic compound, such as a fat, oil, wax, sterol or triglyceride that is insoluble in water but soluble in non-polar organic solvents and is oily to the touch.

As used herein, the term “carbohydrate” means any organic compound, such as a sugar, starch, cellulose or gum, which serves as a major energy source in the diet of animals.

As used herein, the term “protein” means any complex organic macromolecules that contains carbon, hydrogen, oxygen, nitrogen, and usually sulphur and is composed of one or more chains of amino acids.

The microalgal biomass from which the lipid containing products and, optionally carbohydrate containing products and/or protein containing products, are extracted is an aqueous suspension. The aqueous suspension may be prepared by hydrating dried biomass. The hydration may be carried out by contacting the dried microalgal biomass with water for a time and at a temperature sufficient to rehydrate the biomass. For example, the dried biomass may be immersed in water for about 60 minutes to provide the aqueous suspension of microalgal biomass.

Alternatively, the aqueous suspension of microalgal biomass may be a “wet” biomass which has not previously undergone a drying step. As previously mentioned, the drying step that is often used in extracting lipids from microalgae is time and energy intensive and for this reason, the extraction of lipid containing products directly from “wet” biomass may be particularly advantageous.

Alternatively, the aqueous suspension of microalgal biomass may be a concentrated algal paste which has previously undergone a partial drying step.

The aqueous suspension of microalgal biomass comprises about 10% to about 90% by weight of water.

The microalgal biomass may be derived from any suitable microalgae species. Particular microalgae species may be selected based on the particular product(s) to be derived from the biomass. For example, biofuels may be derived from a marine microalga Nanochloropsis sp. The microalgae may be cultivated using conditions known to be suitable for the particular species. For example, Nanochloropsis sp., a marine algal species, may be cultivated in sea water ponds supplemented with suitable media. The microalgae can also be cultivated in photobioreactor systems where many parameters including temperature and light intensity can be controlled under sterile conditions.

The lipid containing products may form an oil layer on top of the aqueous suspension once they are released from the biomass. The lipids can then be physically separated from the aqueous suspension. Optionally, the aqueous suspension may be subjected to centrifugation after the microwave irradiation to assist in separating the oil layer from the aqueous layer and any solid material in the suspension. Optionally, the aqueous suspension may be subjected to extraction with a solvent after the microwave irradiation to extract the lipid containing product from the aqueous layer and any solid material in the suspension. Solvent extraction may be particularly useful when the lipid products are bound in the biomass. Suitable solvents include non aqueous solvents. Suitable non aqueous solvents include non-polar organic liquids. Hydrocarbons, such as hexane or petroleum ethers are suitable non-polar organic liquids for this purpose. Other suitable solvents include esters, ethers, ketones, and nitrated and chlorinated hydrocarbons.

In addition to lipid extraction, carbohydrate materials can also be selectively extracted from the microalgal biomass by decreasing the pH of the aqueous suspension of microalgal biomass. Thus, the present invention provides a method for extracting lipid containing products and carbohydrate containing products from microalgal biomass, the process comprising: (i) providing an aqueous suspension of microalgal biomass; (ii) adjusting the pH of the aqueous suspension to pH<7; (iii) heating the aqueous suspension of microalgal biomass; and (iv) separating the lipid containing products and the carbohydrate containing products from the biomass.

The pH of the aqueous suspension may be lowered using a suitable acid. The acid may be an organic acid or an inorganic acid. In some embodiments, the acid is an inorganic acid. The inorganic acid may be selected from the group consisting of sulfuric acid, nitric acid, hydrochloric acid, and hydrofluoric acid. In some specific embodiments, the acid is sulfuric acid.

The pH of the aqueous suspension may be lowered to less than pH 6. In some embodiments, the pH of the aqueous suspension is lowered to less than about pH 5. In some embodiments, the pH of the aqueous suspension is lowered to less than about pH 4. In some embodiments, the pH of the aqueous suspension is lowered to less than about pH 3. In some embodiments, the pH of the aqueous suspension is lowered to less than about pH 2. In some embodiments, the pH of the aqueous suspension may be lowered to between about 0.5 and about 3. In some embodiments, the pH of the aqueous suspension may be lowered to between about 0.5 and about 2.

The step of heating the aqueous suspension of microalgal biomass may be carried out using any suitable heat source. A number of heat sources are known to the person skilled in the art and can be used for this purpose. Examples include heat baths, autoclaves and microwave ovens. The heating step may be carried out at atmospheric pressure or at a pressure above atmospheric pressure. In the latter case, an autoclave may be used.

We have found that it is particularly advantageous to conduct the heating step by treating the aqueous suspension of microalgal biomass with microwave radiation. One of the advantages of using microwave radiation is a reduction in the time taken for the step.

The step of exposing the aqueous suspension of microalgal biomass to microwave radiation may comprise placing a vessel containing the aqueous suspension of microalgal biomass in a microwave oven. Alternatively, the aqueous suspension of microalgal biomass may be passed through a microwave oven having a continuous flow tube positioned in the microwave oven. The aqueous suspension of microalgal biomass may be exposed to microwave radiation for any period that results in the separation of the lipid containing product from the aqueous suspension. In some embodiment, the microalgal biomass is exposed to microwave radiation for a period of about 1 minute to about 30 minutes. In some embodiments, the microalgal biomass is exposed to microwave radiation for a period of about 10 minutes. The period of time over which the microalgal biomass is exposed to microwave radiation will depend, at least in part, on the output power of the microwave oven.

In some embodiments, the step of exposing the aqueous suspension of microalgal biomass to microwave radiation comprises irradiating the suspension using a first microwave power until the temperature rises to between about 80° C. and about 110° C. and then maintaining the suspension at about 80° C. and about 110° C. for between about a minutes and about 30 minutes using a second microwave power which is lower than the first microwave power. In some specific embodiments, the step of exposing the aqueous suspension of microalgal biomass to microwave radiation comprises irradiating the suspension at a microwave power of about 1000 Watts until the temperature of the suspension rises to about 100° C. and then maintaining the suspension at about 100° C. for about 10 minutes using a microwave power input of about 200 Watts.

After heating as described previously, the lipid containing product may be found in the oil layer on top of the aqueous suspension whereas the carbohydrate containing products can be found in the aqueous solution. If that is the case, the oily layer and the aqueous liquid can be separated from the solid biomass by centrifugation. This provides oil suitable for use either directly or indirectly for fuel purposes and an aqueous solution containing carbohydrates. If necessary, the carbohydrates may be recovered from the aqueous solution using standard techniques, such as solvent evaporation, crystallisation, chromatography, etc. Alternatively, the lipid containing product may be extracted from the aqueous suspension using solvent extraction at a later stage of the process as described in more detail later.

Protein materials can also be selectively extracted from microalgal biomass by increasing the pH of the aqueous suspension of microalgal biomass. Thus, in a third aspect the present invention provides a method for extracting lipid containing products and protein containing products from microalgal biomass, the process comprising: (i) providing an aqueous suspension of the microalgal biomass; (ii) adjusting the pH of the aqueous suspension to pH>7; (iii) heating the microalgal biomass; and (iv) separating the lipid containing products and the protein containing products from the biomass.

The pH of the aqueous suspension may be increased using a suitable base. The base may be an organic base or an inorganic base. In some embodiments, the base is an inorganic base. The inorganic base may be selected from the group consisting of sodium hydroxide, potassium hydroxide, and ammonium hydroxide. In some specific embodiments, the base is sodium hydroxide.

The pH of the aqueous suspension may be increased to greater than pH 8. In some embodiments, the pH of the aqueous suspension is increased to greater than pH 9. In some embodiments, the pH of the aqueous suspension is increased to greater than pH 10. In some embodiments, the pH of the aqueous suspension is increased to greater than pH 11. In some embodiments, the pH of the aqueous suspension is increased to greater than pH 12. In some embodiments, the pH of the aqueous suspension may be increased to between about 10 and about 13.5. In some embodiments, the pH of the aqueous suspension may be increased to between about 11 and about 13.5. In some embodiments, the pH of the aqueous suspension may be increased to between about 12 and about 13.5.

The step of heating the aqueous suspension of microalgal biomass may be carried out using any suitable heat source. A number of heat sources are known to the person skilled in the art and can be used for this purpose. Examples include heat baths, autoclaves and microwave ovens. The heating step may be carried out at atmospheric pressure or at a pressure above atmospheric pressure. In the latter case, an autoclave may be used.

The step of exposing the aqueous suspension of microalgal biomass to microwave radiation may comprise placing a vessel containing the aqueous suspension of microalgal biomass in a microwave oven. Alternatively, the aqueous suspension of microalgal biomass may be passed through a microwave oven having a continuous flow tube positioned in the microwave oven. The aqueous suspension of microalgal biomass may be exposed to microwave radiation for any period that results in the separation of the lipid containing product from the aqueous suspension. In some embodiment, the microalgal biomass is exposed to microwave radiation for a period of about 1 minute to about 30 minutes. In some embodiments, the microalgal biomass is exposed to microwave radiation for a period of about 10 minutes. The period of time over which the microalgal biomass is exposed to microwave radiation will depend, at least in part, on the output power of the microwave oven.

In some embodiments, the step of exposing the aqueous suspension of microalgal biomass to microwave radiation comprises irradiating the suspension using a first microwave power until the temperature rises to between about 80° C. and about 110° C. and then maintaining the suspension at about 80° C. and about 110° C. for between about a minutes and about 30 minutes using a second microwave power which is lower than the first microwave power. In some specific embodiments, the step of exposing the aqueous suspension of microalgal biomass to microwave radiation comprises irradiating the suspension at a microwave power of about 1000 Watts until the temperature of the suspension rises to about 100° C. and then maintaining the suspension at about 100° C. for about 10 minutes using a microwave power input of about 200 Watts.

After heating as described previously, the lipid containing product may be found in the oil layer on top of the aqueous suspension whereas the protein containing products can be found in the aqueous liquid. If that is the case, the oily layer and the aqueous liquid can be separated from the solid biomass by centrifugation. This provides oil suitable for use either directly or indirectly for fuel purposes and an aqueous solution containing proteins. If necessary, the proteins may be recovered from the aqueous solution using standard techniques, such as solvent evaporation, crystallisation, chromatography, etc. Alternatively, the lipid containing product may be extracted from the aqueous suspension using solvent extraction at a later stage of the process as described in more detail later.

The acidic and alkaline extractions may be carried out sequentially. Thus, the pH of the aqueous suspension may be lowered, the suspension heated, the mixture centrifuged to provide an oil layer and aqueous layer containing carbohydrates and a biomass pellet. The biomass pellet can then be suspended in an aqueous solution having a pH>7 to form a second aqueous suspension which may be heated and then the mixture centrifuged to provide a lipid containing layer (if present), a aqueous layer containing proteins and a biomass pellet. The processes may be repeated, as required.

In some embodiments, the method comprises a first extract ion at pH<7 followed by one or more extractions at pH>7 followed by recovery of the lipid containing product. Thus, the present invention provides a process for extracting lipid containing products, carbohydrate containing products, and protein containing products from microalgal biomass, the process comprising:
(i) providing an initial aqueous mixture containing the microalgal biomass;
(ii) adjusting the pH of the initial aqueous mixture to pH<7;
(iii) heating the acidic initial aqueous mixture containing the microalgal biomass to provide a first treated mixture;
(iv) separating the solid and the liquid from the first treated mixture to provide a first solid and a carbohydrate containing liquid;
(v) combining the first solid with an aqueous mixture to form a second aqueous mixture containing microalgal biomass;
(vi) adjusting the pH of the second aqueous mixture to pH>7;
(vii) heating the alkaline second aqueous mixture containing the microalgal biomass to provide a second treated mixture;
(viii) separating the solid and the liquid from the second treated mixture to provide a second solid and a protein containing liquid;
(ix) optionally, repeating steps (v) to (viii) using the second solid to provide a third solid and a further protein containing liquid;
(x) combining the second solid or the third solid with an aqueous mixture to form a final aqueous mixture;
(xi) treating the final aqueous mixture with a solvent;
(xii) Separating the solvent from the final aqueous mixture to provide a solvent containing lipid product.

The pH of the aqueous suspension may be lowered to less than pH 6. In some embodiments, the pH of the aqueous suspension is lowered to less than about pH 5. In some embodiments, the pH of the aqueous suspension is lowered to less than about pH 4. In some embodiments, the pH of the aqueous suspension is lowered to less than about pH 3. In some embodiments, the pH of the aqueous suspension is lowered to less than about pH 2. In some embodiments, the pH of the aqueous suspension may be lowered to between about 0.5 and about 3. In some embodiments, the pH of the aqueous suspension may be lowered to between about 0.5 and about 2. In some embodiments, the pH of the mixture formed in step (ii) is in the range of from about 0.5 to about 2. In some specific embodiments, the pH of the mixture formed in step (ii) is about 1.

The pH of the aqueous suspension may be increased to greater than pH 8. In some embodiments, the pH of the aqueous suspension is increased to greater than pH 9. In some embodiments, the pH of the aqueous suspension is increased to greater than pH 10. In some embodiments, the pH of the aqueous suspension is increased to greater than pH 11. In some embodiments, the pH of the aqueous suspension is increased to greater than pH 12. In some embodiments, the pH of the aqueous suspension may be increased to between about 10 and about 13.5. In some embodiments, the pH of the aqueous suspension may be increased to between about 11 and about 13.5. In some embodiments, the pH of the aqueous suspension may be increased to between about 12 and about 13.5. In some embodiments, the pH of the mixture formed in step (vi) is in the range of from about 11 to about 14. In some specific embodiments, the pH of the mixture formed in step (iv) is about 13.

The steps of heating the acidic or alkaline aqueous mixtures containing the microalgal biomass may be carried out using any suitable heat source. The heating step(s) may be carried out at atmospheric pressure or at above atmospheric pressure. In some embodiments, the steps of heating the acidic or alkaline aqueous mixtures containing the microalgal biomass are carried out by treating the mixtures with microwave radiation.

The step of exposing the aqueous suspension of microalgal biomass to microwave radiation may comprise placing a vessel containing the aqueous suspension of microalgal biomass in a microwave oven. Alternatively, the aqueous suspension of microalgal biomass may be passed through a microwave oven having a continuous flow tube positioned in the microwave oven. The aqueous suspension of microalgal biomass may be exposed to microwave radiation for any period that results in the separation of the lipid containing product from the aqueous suspension. In some embodiment, the microalgal biomass is exposed to microwave radiation for a period of about 1 minute to about 30 minutes. In some embodiments, the microalgal biomass is exposed to microwave radiation for a period of about 10 minutes. The period of time over which the microalgal biomass is exposed to microwave radiation will depend, at least in part, on the output power of the microwave oven.

In some embodiments, the step of exposing the aqueous suspension of microalgal biomass to microwave radiation comprises irradiating the suspension using a first microwave power until the temperature rises to between about 80° C. and about 110° C. and then maintaining the suspension at about 80° C. and about 110° C. for between about a minutes and about 30 minutes using a second microwave power which is lower than the first microwave power. In some specific embodiments, the step of exposing the aqueous suspension of microalgal biomass to microwave radiation comprises irradiating the suspension at a microwave power of about 1000 Watts until the temperature of the suspension rises to about 100° C. and then maintaining the suspension at about 100° C. for about 10 minutes using a microwave power input of about 200 Watts.

The solvent used in the step of treating the final aqueous mixture with a solvent may be any non-aqueous solvent.

The lipid containing products (oils), carbohydrates and proteins derived from the microalgal biomass may be used in a wide range of applications. Microalgal oils are made of different types of fatty acids with many different functions. The neutral lipid, triacylglycerol (TAG) can be used for bio-diesel; the polyunsaturated fatty acids such as omega-3-fatty acids DHA and EPA can be used as nutraceuticals; the membrane lipids may be used as bio-surfactants, and bio-lubricants. If required, the lipid containing products may be further fractionated and/or purified to provide specific lipids for further use.

The microalgal-derived proteins can be used as food or feed protein supplements for both human and animals. Microalgal proteins can be also very important sources of new enzymes for bio-catalysis and biotransformation. Moreover, hydrolysis of these proteins can provide functional polypeptides and oligopeptides that can be used for food applications, nutraceutical products, and pharmaceutical products.

The microalgal-derived carbohydrates can be used as biopolymers, raw sugar for fermentative production of biochemicals, such as bioethanol, lactic acid, antibiotics. The microalgal carbohydrates may be hydrolysed to produce functional polysaccharides or oligo-saccharides that can be used as human nutritional and health products.

The invention is hereinafter described by way of the following non-limiting examples.

### EXAMPLES

The microalgal biomass was obtained from the marine microalga Nanochloropsis sp. Nanochloropsis sp was cultivated in 3000 L raceway ponds outdoors with 20 ppt sea water, supplemented with F/2 media. The pH was generally controlled at 7.9 to 8.2 with 1-5% CO₂. Temperature and illumination were not controlled in the outdoor conditions.

The microalgae could also be cultivated in a photobioreactor where many parameters including temperature and light intensity can also be controlled under sterile conditions.

### Example 1

### Microwave Extraction of Dry Microalgal Biomass Under Acid and Alkaline Conditions

Materials

Dry microalgal biomass, batch number 29 was obtained from South Australian Research and Development Institute (SARDI).

Equipment

Microwave extractions were performed in a Milestone, Start Synth microwave synthesis labstation.

Procedures

Dry biomass (2.5 g) was added to a 50 mL round flat bottom flask. Either 25 mL of 0.5 M H₂SO₄ solution or 25 mL of 0.5 M NaOH was added to the sample. The sample was then incubated at room temperature for about 1 hour and then subjected to microwave irradiation at 100° C. for 2, 5, or 30 minutes.

The sample was then centrifuged at 10,000 g for 10 mins and the top (lipid) layer and middle (carbohydrate and protein) layers were collected separately.

The lipid extract was analysed by gravimetric methods, the carbohydrate extract was analysed by DNS methods, and the protein extract was analysed by the BCA method.

Results

Lipid Extraction

The amount of lipid extracted by the microwave extraction under either the acidic or alkaline condition was difficult to analyse. By collecting 2 mL of the top layer and analysing by gravimetric method, the amount of lipid extracted from 2.5 g dry biomass was less than 10 mg. However, the problem can be solved by large scale extraction.

Optimisation of Microwave Irradiation Time for Carbohydrate and Protein Extraction

Microwave irradiation was carried out for periods of 2 minutes, 5 minutes, 10 minutes and 30 minutes in order to optimise the processing conditions required for extraction of carbohydrates and proteins.

The results are shown in FIG. 1. The alkaline conditions were not suitable for carbohydrate extraction. The carbohydrate content calculated based on 2, 5, 10 and 30 mins extraction were all lower than 0.2%. On the other hand, the carbohydrate content calculated based on 2, 5, 10 and 30 mins extraction under acidic conditions were from about 4% to 5% and the highest value was obtained after 30 minutes irradiation and the lowest value was obtained after 2 minutes irradiation. There was only a small difference between the carbohydrate content calculated based on 5 and 10 minutes of irradiation.

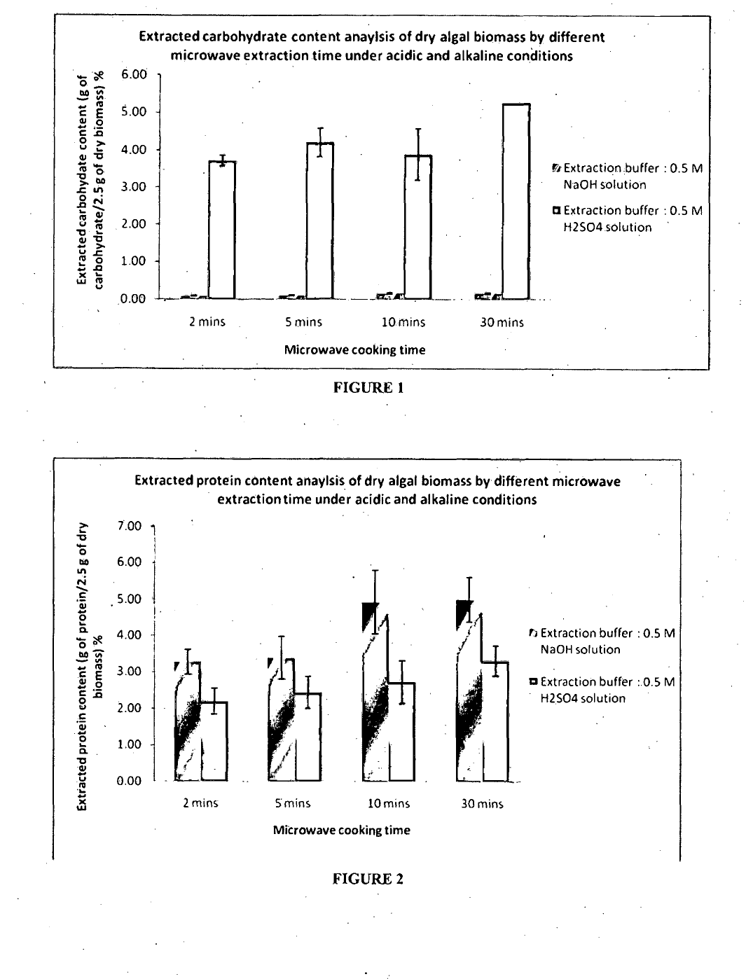

As seen in FIG. 2, for protein extraction alkaline conditions were better than the acidic conditions. The protein content calculated based on 2, 5, 10 and 30 minutes irradiation under alkaline conditions were from 4% to 6% and the highest value was obtained by 10 minutes irradiation. The protein content calculated based on the 2, 5, 10 and 30 minutes irradiation under acidic conditions was from 2% and 3%. The highest value was obtained after 30 minutes irradiation and the second highest value was obtained after 10 mins irradiation.

Furthermore, comparing the values of carbohydrate and protein content obtained after different irradiation times, the variation was small. However, in order to achieve high extraction yields for both carbohydrate and protein and save energy and time, 10 minutes extraction was selected for further experiments.

Repeated Microwave Extraction Under Acidic Conditions

The microwave extraction under acidic conditions was repeated six times to test the efficiency of carbohydrate and protein extraction from the dry microalgal biomass.

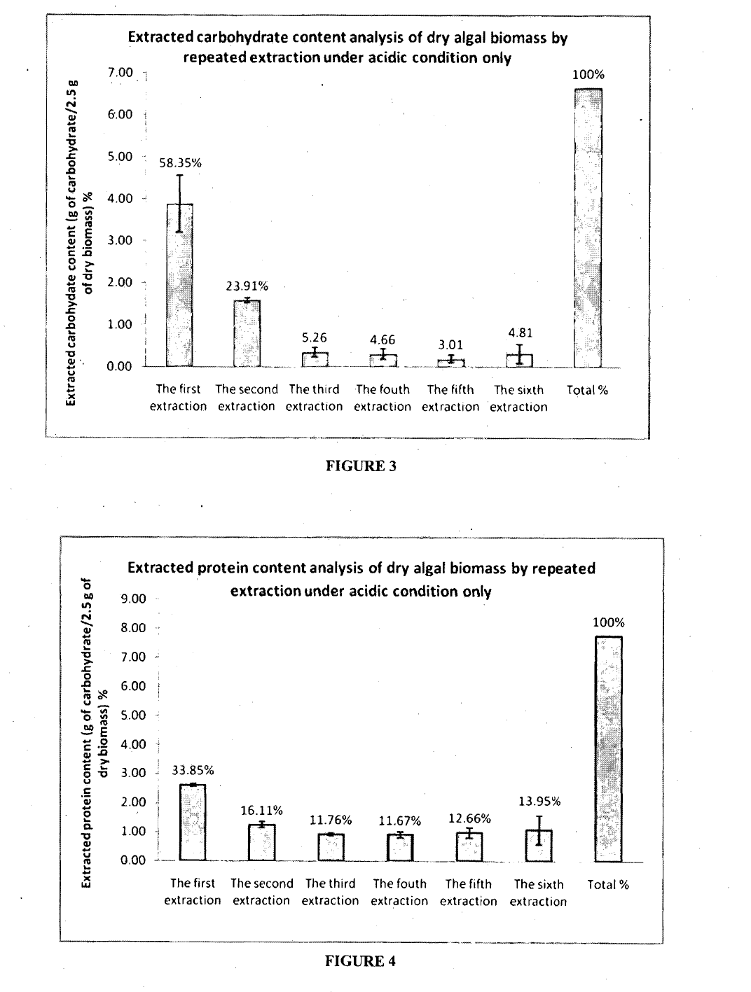

As shown in FIG. 3, after six times repeated acidic extraction, the total carbohydrate extracted from 2.5 g of dry microalgal biomass was about 6.5%. The first extraction recovered about 4% of the carbohydrate from the dry microalgal biomass which is 58.35% of the total extracted carbohydrate, and the second extraction recovered 23.91% of the total extracted carbohydrate. Therefore, about 85% of the carbohydrate can be extracted by the first and second microwave extraction under acidic conditions.

However, the extraction efficiency of protein extraction under acidic conditions was different from the carbohydrate extraction. The protein content of the dry microalgal biomass calculated based on the first extraction was about 2.6% which is 33.85% of the total protein extracted after 6-times repeated extraction. The values obtained from the second to the sixth extractions were similar. This indicates that microwave extraction under acidic conditions might not be efficient enough to extract protein from dry microalgal biomass.

Repeated Microwave Extraction Under Alkaline Conditions

The microwave extraction under alkaline conditions was repeated six times to test the efficiency of carbohydrate and protein extraction from dry microalgal biomass.

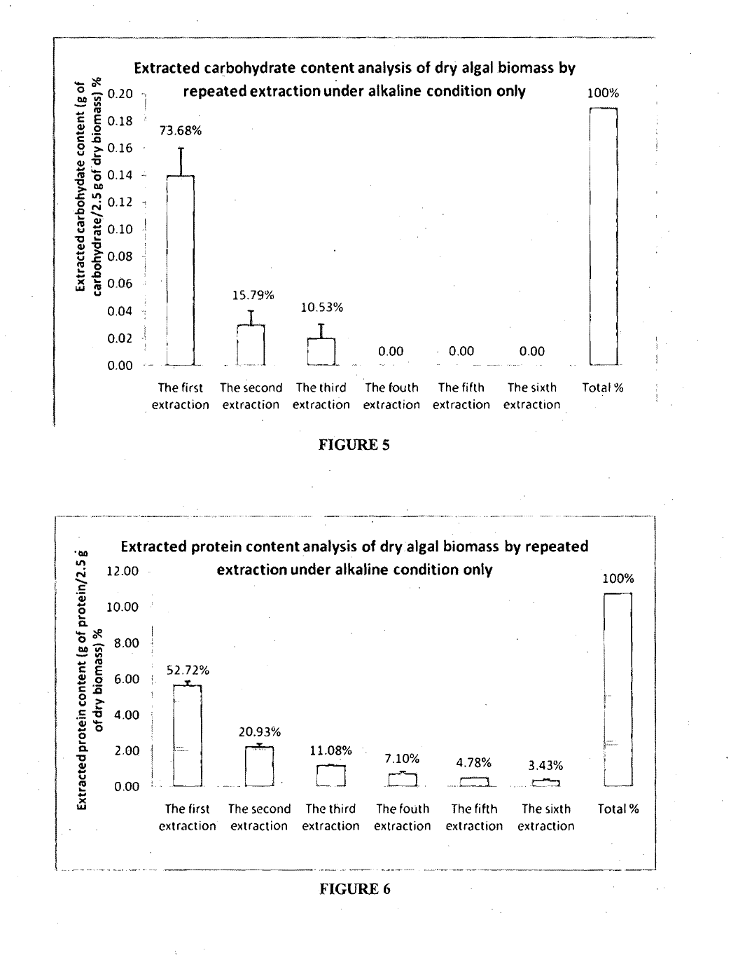

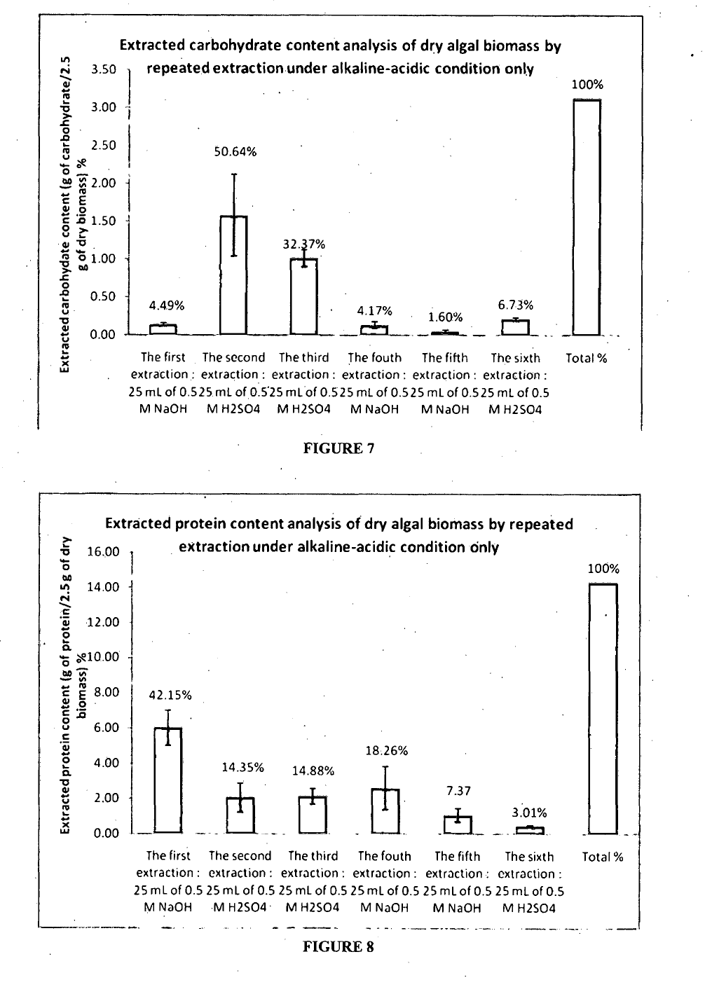

Microwave extraction under alkaline conditions was not suitable for carbohydrate extraction from dry microalgal biomass. After 6 repeated extractions, the total carbohydrate extracted from the dry microalgal biomass was less than 0.2% by weight (g of carbohydrate/g of the dry biomass). However, as shown in FIG. 6, after six consecutive alkaline extractions, the total protein extracted from 2.5 g of dry microalgal biomass was about 0.28 g, 11% of the dry biomass. The first extraction recovered about 52.72% of the total extracted protein and the second extraction recovered 20.93% of the total extracted protein. Therefore, about 74% of the protein can be extracted by the first and second microwave extractions under alkaline conditions.

Repeated Microwave Extraction Under Alkaline to Acidic Conditions

Microwave extraction under alkaline to acidic conditions was repeated six times to test the efficiency of carbohydrate and protein extraction from dry microalgal biomass. The first extraction was under alkaline conditions and the second and third extractions changed to acidic conditions. The fourth and fifth extractions were under alkaline conditions again and the sixth extraction changed to acidic conditions.

The results are shown in FIG. 7. The results of the carbohydrate extractions were different for the alkaline to acidic repeated extractions compared to the acidic and alkaline only extractions. After the first alkaline extraction, the extracted carbohydrate from the dry biomass was less than 0.2%, which is the same as the result obtained from alkaline only extraction. However, after changing to acidic conditions, the second and third extractions showed much lower carbohydrate extraction efficiency. Under the acidic only condition, the first and second extractions provided about 4% and 1.5% carbohydrate. After the alkaline to acidic extraction, the second and third extractions were both under acidic conditions and the carbohydrate content obtained from those two extractions was only about 1.5% and 1%. Furthermore, the total carbohydrate content calculated after six repeated acidic extractions was about 6.5% and the total carbohydrate content calculated after six repeated alkaline to acidic extractions was about 3%.

The protein extraction results are shown in FIG. 8. The total protein content calculated based on the six repeated alkaline to acidic extractions was about 14% which is higher than the result obtained from the six repeated alkaline only extractions. Furthermore, the first alkaline extraction provided about 6% of the dry microalgal biomass and takes account of 42.15% of the total protein extracted. The second and third extractions (acidic) provided about 4% of the dry microalgal biomass in total and it is similar to the first and second acidic only extractions (FIG. 4). More protein was extracted by the fourth extraction (alkaline), compared to the second and third extractions (acidic).

Repeated Microwave Extraction Under Acidic to Alkaline Conditions

Microwave extraction under acidic to alkaline conditions was repeated six times to test the efficiency of carbohydrate and protein extraction from dry microalgal biomass. The first extraction was under acidic conditions, the second and third extractions changed to alkaline conditions, the fourth and fifth extractions were under acidic conditions again, and the sixth extraction changed to alkaline conditions.

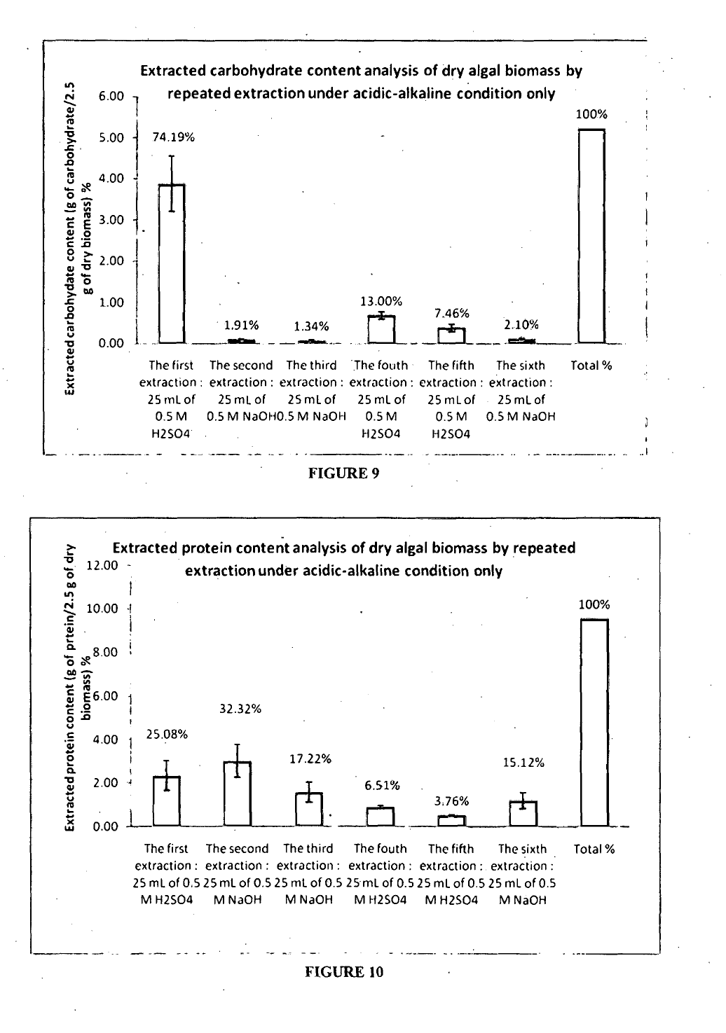

The results are shown in FIG. 9. The performance of carbohydrate extracted from dry biomass by repeated extraction under acidic conditions was similar to the result from the alkaline and acidic only conditions (FIG. 6). The majority of the carbohydrate was extracted by the first, fourth and fifth extraction (acidic), which is about 95% of total carbohydrate extracted by all six extractions. However, even though the second, third and sixth extractions (alkaline) only contributed about 5% of the dry biomass, the actual carbohydrate content calculated by those three extractions is much higher than the result obtained from the alkaline only extractions (FIG. 5).

However, the total protein content calculated based on the six repeated acidic to alkaline extractions was about 10% which is higher than the result obtained from six repeated alkaline only extractions (FIG. 6) and similar to the result from the alkaline to acidic extractions (FIG. 8). Furthermore, the alkaline extractions showed better performance than the acidic extractions for protein. About 65% of extracted protein was obtained by the second, third and sixth extractions (alkaline).

Test of Lipid, Carbohydrate and Protein Recovery by Spiking the Dry Biomass with Known Amounts of Lipid, Carbohydrate and Protein

The dry microalgal biomass was spiked with 4 mg of standard protein BSA, 125 mg of glucose and 0.260 g of canola oil. Multiple extractions were then carried out using the regime “acidic-alkaline-alkaline-acidic-acidic-alkaline”. A control extraction without added protein, glucose or oil was also carried out.

The lipid recovery was still difficult to measure. Even with 0.26 grains of spiked oil, the amount of lipid extracted after the treatment was still too small for analysis.

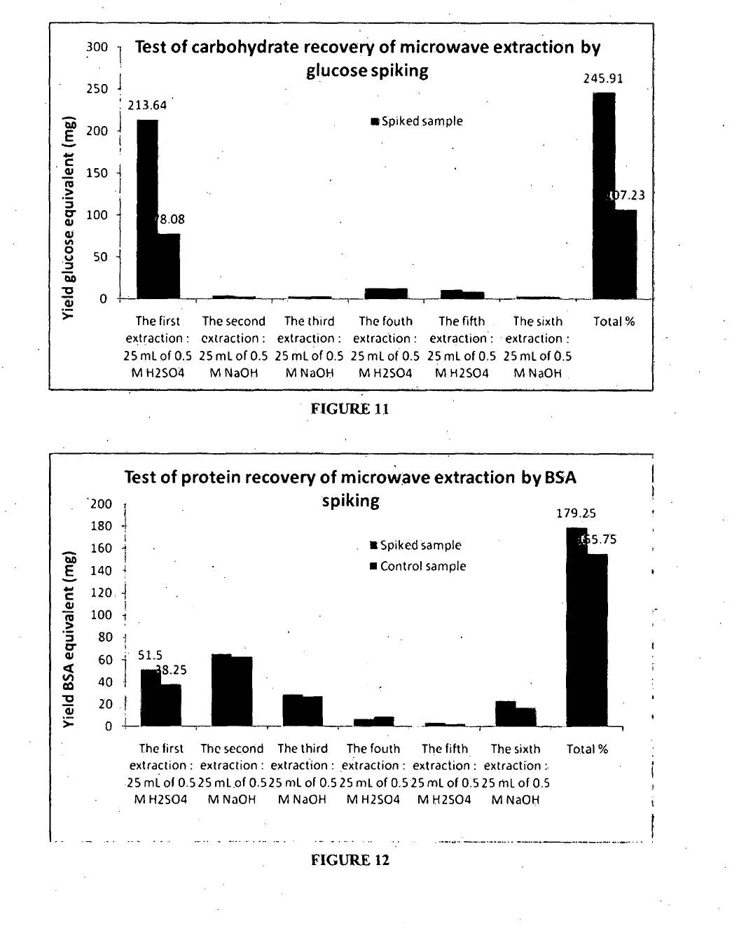

For the carbohydrate recovery, the first acid extraction of spiked material yielded 213.64 mg glucose equiv. This was 135.56 mg greater than the control extraction which yielded 78.08 mg (FIG. 11). The value is quite close to the spiking glucose, 125 gram. This result provides evidence that all of the added glucose was recovered in the first acid extraction. The added BSA protein and canola oil may have contributed to the extra 10 mg of glucose equiv which was recovered from the spiked biomass.

Note that the total glucose extracted under the standard conditions (as used for the control) was 107.23 mg which was lower than the value of 131.05 mg which was the average of the previous runs (FIG. 9) under the same conditions.

For the protein recovery, the first acid extraction of spiked material yielded 51.5 mg BSA equiv. This was 13.25 mg greater than the control extraction which yielded 38.25 mg (FIG. 12). However, compare to the amount of BSA added before the extraction, 4 mg, the test value was much higher. Furthermore, according to the total protein recovered from the six extractions, the spiked sample showed 23.5 g of BSA equiv more than the control sample. It could due to the amount of spiking protein was too small to cover the variation between the repeated experiments.

### Example 2

### Scale-Up of Extractions Under Selected Conditions

In order to recover most of the carbohydrate and protein during the microwave extraction, the acidic to alkaline (acidic-alkaline-alkaline-acidic-acidic-alkaline) repeated condition was selected. According to FIGS. 9 and 10, the total extracted carbohydrate content of the dry microalgal biomass through six extractions was up to 5% and the extracted protein content of the dry microalgal biomass through six extractions was up to 10%. Furthermore, according to FIG. 9, the majority of the extracted carbohydrate is able to be extracted through the first acid extraction and only a minimum amount of extracted carbohydrate was provided from the second and third extractions. For the protein, the first acidic extraction contributes about 25% of the total extracted protein and the second extraction and third extraction provided about 30% and 20% of total extracted protein, respectively. Therefore, the acidic to alkaline condition not only maximises the carbohydrate and protein extraction efficiency, but also enables ready product separation. After the first acidic extraction, the majority of extracted carbohydrate is removed. Therefore, extracts from the second and third alkaline extractions are almost carbohydrate free.

Materials

Dry microalgal biomass, batch number 29 was obtained from South Australian Research and Development Institute (SARDI).

Equipment

Microwave extractions were performed in a Milestone, Start Synth microwave synthesis labstation.

Procedures

Dry biomass (25 g) was added to a 500 mL flat bottom flask. 0.5 M H₂SO₄ (250 mL) was added and the sample was incubated at room temperature for about 1 hour. The sample was then irradiated in the microwave oven at 100° C. for 10 mins.

The mixture was then centrifuged at 10,000 g for 10 mins and the top (lipid) layer and middle (carbohydrate and protein) layers were separately collected to provide first extraction products.

The pellet was re-suspended in 250 mL of 0.5 M NaOH and the microwave irradiation; centrifugation and product separation process was repeated to provide second extraction products.

The pellet was again re-suspended in 250 mL of 0.5 M NaOH and the microwave irradiation; centrifugation and product separation process was repeated to provide third extraction products.

The pellet was again re-suspended in 250 mL of 0.5 M H₂SO₄ and the microwave irradiation, centrifugation and product separation process was repeated to provide fourth extraction products.

The pellet was again re-suspended in 250 mL of 0.5 M H₂SO₄ and the microwave irradiation, centrifugation and product separation process was repeated to provide fifth extraction products.

Finally, the pellet was again re-suspended in 250 mL of 0.5 M NaOH and the microwave irradiation; centrifugation and product separation process was repeated to provide sixth extraction products.

The lipid extract was analysed by the gravimetric method, the carbohydrate extract was analysed by the DNS method and the protein extract was analysed by the BCA.

Results

Lipid Extracts

No lipid layer was observed after each extraction. Therefore, the supernatants of each extraction were collected and combined together so as to increase the total amount of lipid in the extract. However, no lipid layer was observed after standing the sample at room temperature overnight. A considerable amount of biomass was floating in the sample and it was presumed that this was not making it possible to observe the extracted lipid. Therefore, a 100 mL sample was collected from the top layer of the sample and the lipid analysis by the gravimetric methods was carried out on this material.

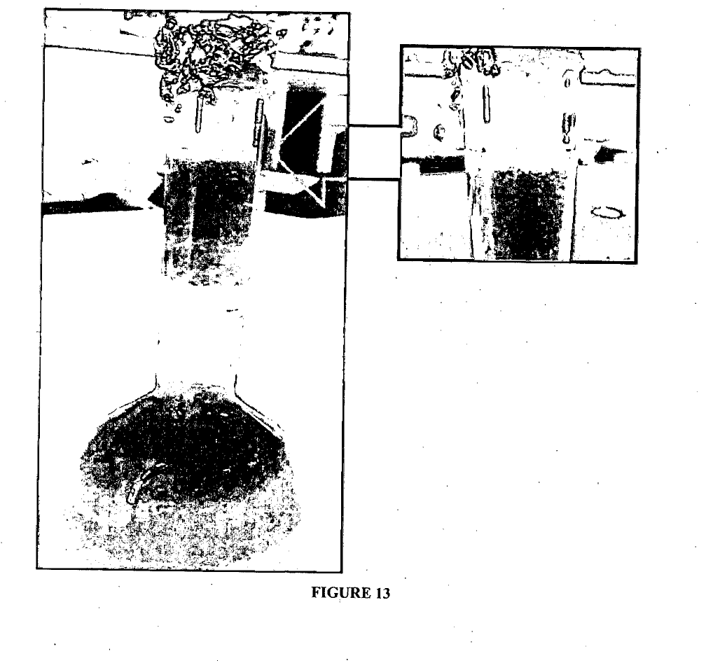

The invisibility of the extracted lipid could also be caused by the shape of the container. In a conventional beaker, it is hard to recognise a small amount of oil on the top of large amount of water. Therefore, in order to concentrate the extracted oil without reducing the amount of water in the sample a volumetric flask was used. The extract which might contain lipid was place in an appreciated volumetric flask, according to the volume of the sample. After overnight standing, a small amount of oil showed on the wall of the flask (FIG. 13).

Carbohydrate and Protein Extracts

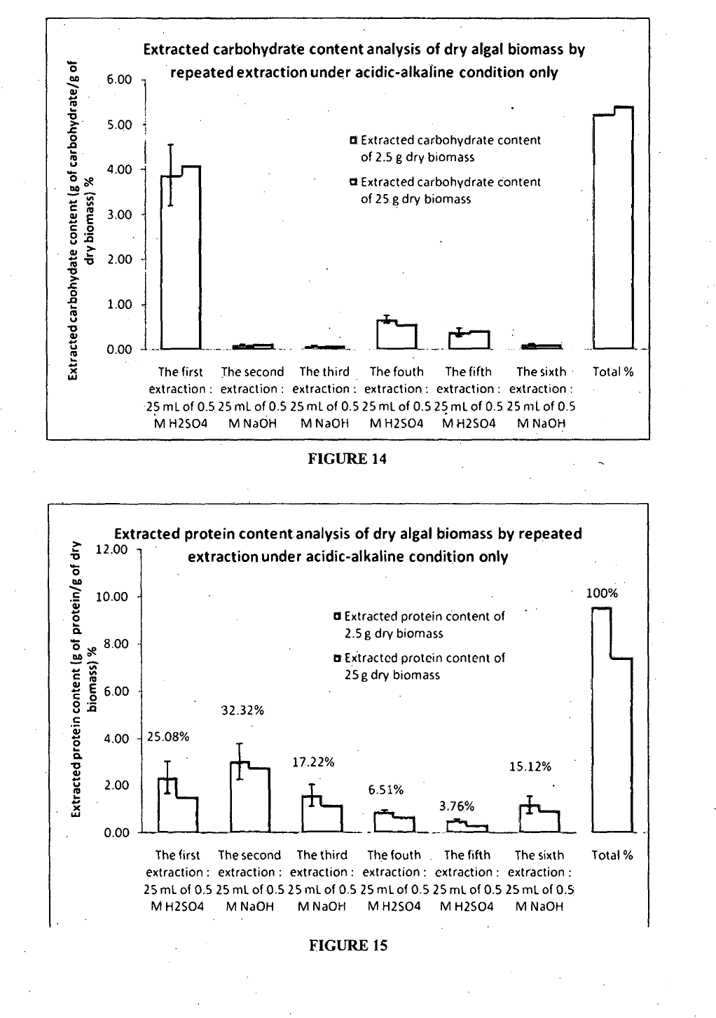

The results of the larger scale carbohydrate extraction were comparable to the small scale extraction (FIG. 14). Therefore, scale up of the carbohydrate extraction was successful. However, as shown in FIG. 15, the protein extraction efficiency on the larger scale was similar to that of the small scale extraction.

### Example 3

### Extraction of a Different Batch of Dry Microalgal Biomass

Materials

Microalgal biomass was obtained from South Australian Research and Development Institute (SARDI) and oven dried at 60 to 80° C.

Equipment

Microwave extractions were performed in a Milestone, Start Synth microwave synthesis labstation.

Procedures

Microalgal powder (2.5 g) was added to a 50 mL flat bottom flask (in duplicate). 0.5 M H₂SO₄ (25 mL) was added and the sample was mixed and then incubated at room temperature for about 1 hour to rehydrate the biomass. The pH of the sample was measured and the sample was then processed in the microwave oven under the following conditions:
Each sample is irradiated at microwave power of 1000 Watt until the sample temperature rises to 100° C. (it usually takes about 30 seconds for a sample volume of 25 mL);
The sample is maintained at 100° C. for 10 mins with 200 W microwave power input.

The sample was then cooled in a water bath for 3 to 5 mins, following which it was transferred into a centrifuge tube and centrifuged at 10,000 g for 10 mins.

The supernatant was then transferred into a 25 mL volumetric cylinder to measure the volume. 1 mL of the supernatant was transferred into a 1.5 mL centrifuge tube for carbohydrate and protein analysis.

The pellet obtained after the first extraction was suspended in 25 mL of 0.5 M NaOH solution and a second extraction which was a repeat of the process of the first extraction was carried out.

The pellet obtained after the second extraction was then suspended in 25 mL of 0.5 M NaOH solution and a third extraction which was a repeat of the process of the first extraction was carried out.

The pellet obtained after the third extraction was then suspended in 25 mL of 0.5 M H₂SO₄ solution and a fourth extraction which was a repeat of the process of the first extraction was carried out.

The pellet obtained after the fourth extraction was the suspended in 25 mL of 0.5 M H₂SO₄ solution and a fifth extraction which was a repeat of the process of the first extraction was carried out.

The pellet obtained after the fifth extraction was suspended in 25 mL of 0.5 M NaOH solution and a sixth extraction which was a repeat of the process of the first extraction was carried out.

Results

As shown in table 1, the pH varied from each extraction under the different conditions. The pH of the first extraction was 1.2. This is because the biomass was harvested under the strong alkaline conditions and part of the acid added was neutralised by the alkaline biomass. After the first extraction, by adding 0.5 M NaOH solution as the second and third extraction buffer, the pH raised to 13. The fourth and fifth extractions were under acidic conditions and the pH dropped to about 0.7 which was significantly lower than the pH of the first extraction. Finally, the pH of the sixth extraction increased to 13.6 by adding 0.5 M NaOH solution.

The volume of supernatant collected after each extraction varied. After the first extraction, 22.5 mL of supernatant was collected. This is about 2.5 mL less than the volume of extraction buffer added for the extraction. As the first extraction started from dry biomass, part of the extraction buffer will have integrated with the biomass in the pellet. From the second to the sixth extraction, the volumes of the supernatant were close to the volume added.

TABLE 1
pH value and volume of the supernatant of each extraction process

| | | | | | Supernatant | Supernatant | Mean |
|---|---|---|---|---|---|---|---|
| | | | | | volume (mL) | volume (mL) | supernatant |
| | Condition | pH 1 | pH 2 | Mean pH | 1 | 2 | volume (mL) |
| 1 | 0.5M | 1.25 | 1.2 | 1.23 | 22.5 | 22.5 | 22.5 |
| | H2SO4 | | | | | | |
| 2 | 0.5M | 13.2 | 13.25 | 13.23 | 23.5 | 24 | 23.75 |
| | NaOH | | | | | | |
| 3 | 0.5M | 13.25 | 13.26 | 13.26 | 25 | 26 | 25.5 |
| | NaOH | | | | | | |
| 4 | 0.5M | 0.7 | 0.8 | 0.75 | 24 | 24 | 24 |
| | H2SO4 | | | | | | |
| 5 | 0.5M | 0.67 | 0.62 | 0.65 | 26 | 26 | 26 |
| | H2SO4 | | | | | | |
| 6 | 0.5M | 13.01 | 13.3 | 13.16 | 26.5 | 25 | 25.75 |
| | NaOH | | | | | | |

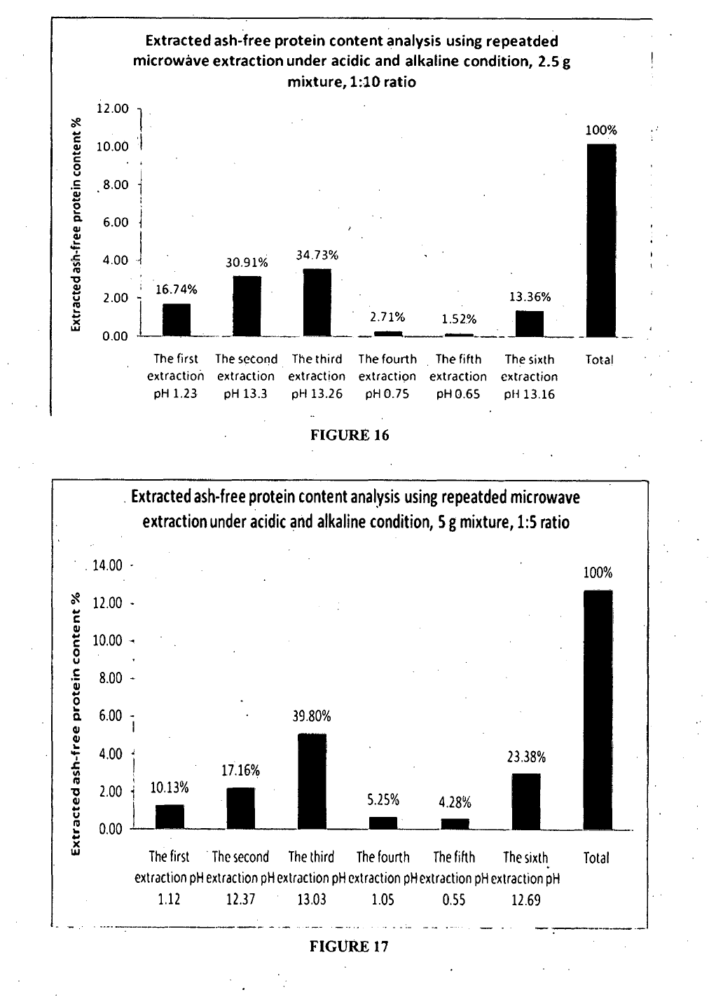

As shown in FIG. 16, after six repeated microwave extractions, the total amount of extracted protein was 11% of the AFDW of the biomass used in the experiment. Compared to the acidic conditions, the first, fourth, fifth extractions, the second and third extractions which are under the alkaline conditions showed higher protein extraction yields. Furthermore, the extracted protein yield obtained from the fourth and fifth extractions (acidic conditions) were low. This may be because most of the extractable proteins were extracted after the first acidic extraction.

### Example 4

### Reduction of the Total Amount of Extraction Buffer Used by Increasing the Amount of Biomass from 2.5 Gram to 5 Gram (the Biomass and Extraction Buffer Ratio to 1:5)

Materials

Microalgal biomass was obtained from South Australian Research and Development Institute (SARDI) and oven dried at 60 to 80° C.

Equipment

Microwave extractions were performed in a Milestone, Start Synth microwave synthesis labstation.

Procedures

Microalgal powder (5 g) was added to a 50 mL flat bottom flask (in duplicate). 1 M H₂SO₄ (25 mL) was added and the sample was mixed and then incubated at room temperature for about 1 hour to rehydrate the biomass. The pH of the sample was measured and the sample was then processed in the microwave oven under the following conditions:
Each sample is irradiated at microwave power of 1000 Watt until the sample temperature rises to 100° C. (it usually takes about 30 seconds for a sample volume of 25 mL);
The sample is maintained at 100° C. for 10 mins with 200 W microwave power input.

The sample was then cooled in a water bath for 3 to 5 mins, following which it was transferred into a centrifuge tube and centrifuged at 10,000 g. for 10 mins.

The supernatant was then transferred into a 25 mL volumetric cylinder to measure the volume. 1 mL of the supernatant was transferred into a 1.5 mL centrifuge tube for carbohydrate and protein analysis.

The pellet obtained after the first extraction was suspended in 25 mL of 0.5 M NaOH solution and a second extraction which was a repeat of the process of the first extraction was carried out.

The pellet obtained after the second extraction was then suspended in 25 mL of 0.5 M NaOH solution and a third extraction which was a repeat of the process of the first extraction was carried out.

The pellet obtained after the third extraction was then suspended in 25 mL of 0.5 M H₂SO₄ solution and a fourth extraction which was a repeat of the process of the first extraction was carried out.

The pellet obtained after the fourth extraction was the suspended in 25 mL of 0.5 M H₂SO₄ solution and a fifth extraction which was a repeat of the process of the first extraction was carried out.

The pellet obtained after the fifth extraction was suspended in 25 mL of 0.5 M NaOH solution and a sixth extraction which was a repeat of the process of the first extraction was carried out.

Results

TABLE 2
The pH value and volume of the supernatant of each extraction
process (biomass and extraction buffer ratio 1:5)

| | | | | | Supernatant | Supernatant | Mean |
|---|---|---|---|---|---|---|---|
| | | | | | volume (mL) | volume (mL) | supernatant |
| | Condition | pH 1 | pH 2 | Mean pH | 1 | 2 | volume (mL) |
| 1 | 1M | 1.08 | 1.15 | 1.12 | 19 | 21 | 20 |
| | H₂SO₄ | | | | | | |
| 2 | 0.5M | 12.5 | 12.24 | 12.37 | 22 | 23 | 22.5 |
| | NaOH | | | | | | |
| 3 | 0.5M | 13 | 13.05 | 13.03 | 22 | 26 | 24 |
| | NaOH | | | | | | |
| 4 | 0.5M | 1 | 1.1 | 1.05 | 27 | 28 | 27.5 |
| | H₂SO₄ | | | | | | |
| 5 | 0.5M | 0.6 | 0.5 | 0.55 | 25 | 22 | 23.5 |
| | H₂SO₄ | | | | | | |
| 6 | 0.5M | 12.7 | 12.67 | 12.69 | 27 | 25 | 26 |
| | NaOH | | | | | | |

Even though the amount of extraction buffer used did not change, in order to increase the biomass and extraction biomass ratio from 1:10 to 1:5, the amount of biomass used was doubled. Therefore, to keep the pH of the first extraction to around 1, instead of using 0.5 M H₂SO₄ solution, 25 mL of 1 M H₂SO₄ solution was added to 5 g of dry biomass. Beside the first extraction, the same extraction buffers were used for the second to the sixth extractions. The pH of each extraction was either about 0.5 to 1 or about 12 to 13.

Compared to the extraction with the 1:10 biomass and buffer ratio, more volume loss occurred after the first extraction with 1:5 biomass and buffer ratio. There was only 20 mL of the supernatant which was collected after the first extraction, thereby indicating that 5 mL of the extraction buffer was incorporated into the biomass. The volume of supernatant collected from the second to the sixth extraction was still around 25 mL.

Results

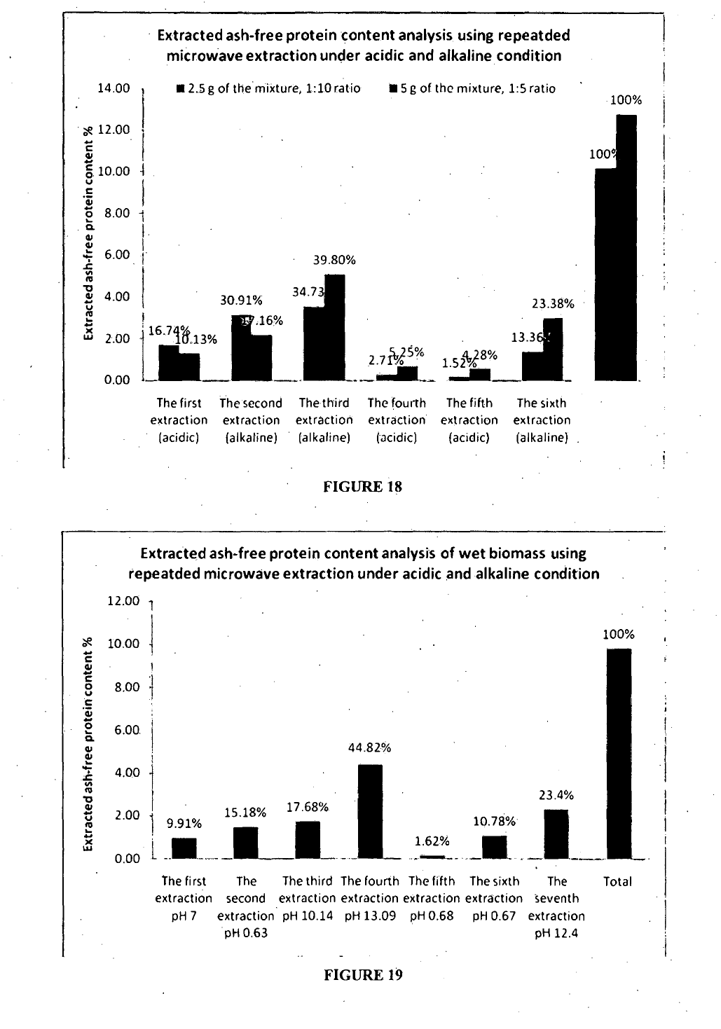

As shown in FIG. 17, after six repeated microwave extractions, the total amount of extracted protein was 13% of the AFDW of the biomass used. Again, the alkaline conditions still showed much better yield of extracted protein compared to the acidic conditions. Compared to the protein extraction yield obtained from extractions with 1:10 biomass and buffer ratio, the protein extraction yield with 1:5 biomass and buffer ratio was higher (FIG. 18), except for the first and the second extractions.

### Example 5

### Microwave Extraction of Wet Microalgal Biomass Directly

Materials

Microalgal biomass was obtained from South Australian Research and Development Institute (SARDI).

Equipment

Microwave extractions were performed in a Milestone, Start Synth microwave synthesis labstation.

Procedures

Wet biomass (25 g) was added to a 50 mL flat bottom flask (in duplicate). Each sample was then processed in the microwave oven under the following conditions:
Each sample is irradiated at microwave power of 1000 Watt until the sample temperature rises to 100° C. (it usually takes about 30 seconds for a sample volume of 25 mL);
The sample is maintained at 100° C. for 10 mins with 200 W microwave power input.

The sample was then cooled in a water bath for 3 to 5 mins, following which it was transferred into a centrifuge tube and centrifuged at 10,000 g for 10 mins.

The supernatant was then transferred into a 25 mL volumetric cylinder to measure the volume. 1 mL of the supernatant was transferred into a 1.5 mL centrifuge tube for carbohydrate and protein analysis.

The pellet obtained after the first extraction was suspended in 12 mL of 1 M H₂SO₄ and a second extraction which was a repeat of the process of the first extraction was carried out.

The pellet obtained after the second extraction was suspended in 15 mL of 0.5 M NaOH and a third extraction which was a repeat of the process of the first extraction was carried out.

The pellet obtained after the third extraction was then suspended in 13 mL of 0.5 M NaOH and a fourth extraction which was a repeat of the process of the first extraction was carried out.

The pellet obtained after the fourth extraction was then suspended in 12 mL of 1 M H₂SO₄ and a fifth extraction which was a repeat of the process of the first extraction was carried out.

The pellet obtained after the fifth extraction was the suspended in 16 mL of 1 M H₂SO₄ and a sixth extraction which was a repeat of the process of the first extraction was carried out.

The pellet obtained after the sixth extraction was suspended in 17 mL of 0.5 M NaOH and a seventh extraction which was a repeat of the process of the first extraction was carried out.

Results

TABLE 3
pH value and volume of the supernatant of
each extraction process (wet biomass)

| | | | | Volume of | Volume of |
|---|---|---|---|---|---|
| | | | | extraction buffer | supernatant |
| | Conditions | | pH | added (mL) | collected (mL) |
| 1 | no treatment | | 7 | 0 | 8 |
| 2 | 1 | M H2SO4 | 0.63 | 12 | 15 |
| 3 | 0.5 | M NaOH | 10.14 | 15 | 13 |
| 4 | 0.5 | M NaOH | 13.09 | 13 | 12 |
| 5 | 1 | M H2SO4 | 0.68 | 12 | 16 |
| 6 | 1 | M H2SO4 | 0.67 | 16 | 17 |
| 7 | 0.5 | M NaOH | 12.4 | 17 | 17 |

In order to apply the optimised microwave extraction technique to wet microalgal biomass, the method described in the previous examples was modified. First of all, the wet biomass contains 82.1% water and, therefore, without any adjustment the dry matter and water is close to 1 to 5, which is similar to the optimised conditions for the extraction method using dry microalgal biomass. However, it is difficult to adjust the pH of the wet biomass. Therefore, the first extraction was carried out without adjusting the pH. After microwave irradiation and centrifugation, 8 mL of water was separated from the biomass. It was then possible to adjust the pH of the biomass for the second extraction. The volume of extracts after each extraction was measured in order to calculate the amount of extracted protein and carbohydrate after each extraction and also calculate the volume of extraction buffer to be added for subsequent extraction.

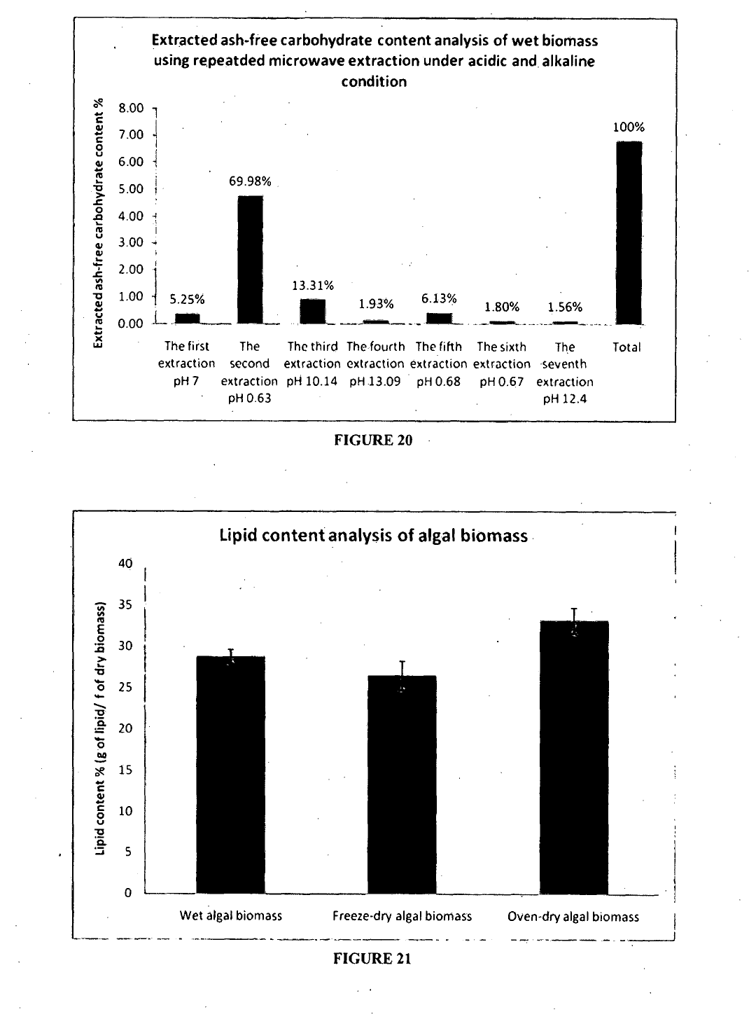

As shown in FIGS. 19 and 20, the total protein and carbohydrate recovery from each extraction was similar to that obtained using dry microalgal biomass. The total extracted protein recovery was about 10% of the total dry weight of the biomass used and the total extracted carbohydrate recovery was about 7% of the total dry weight of the biomass used. Furthermore, the volume of extraction buffer added to each extraction was much less than the volume used to extract dry biomass. Therefore, the concentration of protein and carbohydrate of each extract was much higher. As a comparison of protein and carbohydrate extraction efficiency between using dry and wet microalgal biomass, it results in a similar extraction efficiency for both protein and carbohydrate extraction. Furthermore, by using wet microalgal biomass directly, there is no drying process involved. This is advantageous because it means that after the microalgal biomass is harvested using a centrifuge, the wet paste thus obtained can be processed directly without drying it.

### Example 6

### Determination of the Moisture Content of the Wet Algal Biomass Using the Freeze-Dry Method

Materials

Frozen wet algal biomass was received from SARDI on Jun. 6, 2011.

Procedures

Weigh about 200 g of the wet biomass in a 1 L glass beaker

Actual weight: 200.594 g

Weigh the empty beaker: 81.980 g

Allow the frozen biomass melt at room temperature for 30 mins and refreeze it inside the beaker use liquid nitrogen to form a thin ice shell

Freeze-dry the biomass for 24 hours

Weigh the beaker+dry biomass: 134.252 g

Results

The weight of dry matter was 52.272 g. The dry matter content of the wet algal biomass was 26.06%. The moisture content of the wet biomass was 73.94%.

### Example 7

### Determination of the Moisture Content of the Wet Algal Biomass Using the Oven Dry Method

Materials

Frozen wet algal biomass was received from SARDI on Jun. 6, 2011.

Procedures

Weigh about 50 g of the frozen wet algal biomass in a small glass beaker. Actual weight of the biomass: 59.139 g

Weigh the beaker: 47.690 g

Dry the biomass inside the beaker at 60° C. for 96 hours

Weigh the dry biomass+beaker: 64.1174 g

Results

The weight of dry matter was 16.737 g. The dry matter content of the wet algal biomass was 28.30%. The moisture content of the wet biomass was 71.70%.

### Example 8

### Determination of the Lipid Content of Wet, Freeze Dried and Oven Dried Algal Biomass

Materials

Frozen wet algal biomass was received from SARDI on Jun. 6, 2011. Freeze-dried algal biomass and oven dried algal biomass were prepared according to the methods set out in Examples 6 and 7, respectively.

Equipment

Centrifugation was carried out using an Allegra x-12R centrifuge (Backman Coulter) using a FX6100 rotor (Backman Coulter) and chloroform resistant centrifuge tubes (50 mL).

The vacuum evaporator used was a Benchtop K, VirTis and the freeze-drier was a Labconco.

Procedures
Take about 6 g of frozen wet algal biomass and melt at RT for 30 mins
Weigh about 2.5 of the wet algal biomass into a 50 mL centrifuge tube (in duplicates)
Actual weight: a. 2.5819 g
   b. 2.5305 g
Weigh about 0.5 g of freeze-dry algal biomass into a 50 mL centrifuge tube (in duplicates)
Actual weight: a. 0.4978 g
   b. 0.5197 g
Weigh about 0.5 g of oven-dry algal biomass into a 50 mL centrifuge tube (in duplicates)
Actual weight: a. 0.4962 g
   b. 0.5185 g
Add 17.8 mL of solvent (methanol:chloroform:water 2:1:0.8) to wet biomass samples
Add 22.8 mL of solvent (methanol:chloroform:water 2:1:0.8) to freeze-dry and oven dry biomass samples
Vortex all samples and incubate all samples at RT for 1 hours (with manual shaking every 5 mins)
Add additional 6 mL of chloroform and 6 mL of water to each sample and vortex
Centrifuge all samples at 3000 g for 5 mins to achieve phase separation
Collect 6 mL of the bottom chloroform layer into a pre-weighed glass test tube
Weight of each tube:
Wet algal biomass: a. 11.5835 g
   b. 11.2718 g
Freeze-dry algal biomass: a. 11.6648 g
   b. 11.5558 g
Oven-dry algal biomass: a. 11.5857 g
   b. 11.5481 g
Evaporate the chloroform use vacuum evaporator for 6 hours
Weigh the tube+lipid extract

Results

The results are shown in FIG. 21.

### Example 9

### Lipid Extract Profile Analysis of the Wet, Freeze Dry and Oven Dry Algal Biomass

Materials

The lipid extracts were obtained from wet, freeze-dry and oven dry algal biomass.

Procedures

Lipid profile analysis was conducted using gas chromatography by NCRIS. The results are shown in Table 4.

TABLE 4
Lipid profile of biomass and extracts

| | | | | | | Hexane |
|---|---|---|---|---|---|---|
| | | | | Leftover | Leftover | extracted |
| | | | | biomass | biomass | oil from |
| | | Freeze- | | after | after | microwave |
| | Wet | dry | Oven-dried | microwave | microwave | treated |
| Fatty acid | biomass | biomass | biomass | treatment 1 | treatment 2 | biomass |
| 8:0 | 0.0 | 0.0 | 0.0 | 0.0 | 0.0 | 0.0 |
| 10:0 | 0.0 | 0.0 | 0.0 | 0.0 | 0.0 | 0.0 |
| 12:0 | 0.0 | 0.0 | 0.0 | 0.0 | 0.0 | 0.0 |
| 14:0 | 0.5 | 0.6 | 0.6 | 0.5 | 0.4 | 0.0 |
| 16:0 | 23.8 | 24.7 | 24.7 | 24.7 | 23.3 | 28.3 |
| 18:0 | 0.6 | 0.7 | 0.7 | 0.6 | 0.7 | 0.0 |
| 20:0 | 0.1 | 0.1 | 0.1 | 0.1 | 0.1 | 0.0 |
| 22:0 | 0.0 | 0.0 | 0.0 | 0.0 | 0.0 | 0.0 |
| 24:0 | 0.0 | 0.0 | 0.0 | 0.0 | 0.0 | 0.0 |
| Total Saturated | 25.1 | 26.2 | 26.2 | 26.0 | 24.5 | 28.3 |
| Fatty Acids | | | | | | |
| 16:1n-7 | 6.2 | 5.8 | 5.8 | 5.2 | 4.5 | 36.0 |
| 18:1n-7 | 27.7 | 29.6 | 29.6 | 32.4 | 38.9 | 13.0 |
| 18:1n-9 | 2.7 | 2.8 | 2.8 | 2.4 | 1.7 | 0.0 |
| 20:1n-9 | 0.5 | 0.6 | 0.6 | 0.7 | 0.7 | 0.0 |
| Total | 37.1 | 38.9 | 38.9 | 40.6 | 45.8 | 49.0 |
| Monounsaturated | | | | | | |
| Fatty Acids | | | | | | |
| Total n-9 | 3.2 | 3.5 | 3.5 | 3.1 | 2.4 | 0.0 |
| Total n-7 | 33.9 | 35.4 | 35.4 | 37.5 | 43.4 | 49.0 |
| 18:2n-6 | 8.3 | 0.0 | 0.0 | 8.1 | 7.8 | 0.0 |
| 20:4n-6 | 0.4 | 0.1 | 0.1 | 0.2 | 0.2 | 0.0 |
| Total n-6 | 8.8 | 0.1 | 0.1 | 8.2 | 8.0 | 0.0 |
| 16:3n-4 | 0.2 | 0.2 | 0.2 | 0.2 | 0.1 | 0.0 |
| 18:3n-4 | 0.0 | 0.0 | 0.0 | 0.0 | 0.0 | 0.0 |
| Total n-4 | 0.2 | 0.2 | 0.2 | 0.2 | 0.2 | 0.0 |
| 18:3n-3 | 15.4 | 14.2 | 14.2 | 13.6 | 11.0 | 0.0 |
| 18:4n-3 | 0.2 | 0.1 | 0.1 | 0.1 | 0.1 | 0.0 |
| 20:4n-3 | 0.0 | 0.0 | 0.0 | 0.0 | 0.0 | 0.0 |
| 20:5n-3 | 1.1 | 0.9 | 0.9 | 0.8 | 0.7 | 4.7 |
| 22:5n-3 | 0.0 | 0.0 | 0.0 | 0.0 | 0.0 | 0.0 |
| 22:6n-3 | 0.8 | 0.9 | 0.9 | 0.8 | 0.9 | 0.8 |
| Total n-3 | 17.4 | 16.1 | 16.1 | 15.4 | 12.7 | 5.5 |
| Unknowns | 11.1 | 9.7 | 9.7 | 9.2 | 8.4 | 12.2 |

### Example 10

### Total Carbohydrate Content Analysis of the Algal Biomass

Materials

Freeze dried algal biomass was prepared according to Example 6. Phenol reagent was prepared as required (phenol:MilliQ water 4:1 w/v).

Procedures

A standard curve was prepared as follows:
Prepare 0.1 g/L glucose stock solution by dissolving 1 mg of glucose into 100 mL of MilliQ water.
Prepare glucose standards in large glass test tubes as detailed in the table below:

| Volume glucose stock | | |
|---|---|---|
| solution (at 0.1 g/L) (ml) | MilliQ water (ml) | Glucose concentration (g/l) |
| 0.00 | 2.00 | 0.00 |
| 0.20 | 1.80 | 0.01 |
| 0.40 | 1.60 | 0.02 |
| 0.60 | 1.40 | 0.03 |
| 0.80 | 1.20 | 0.04 |
| 1.00 | 1.00 | 0.05 |

Prepare phenol reagent (80% w/w in MilliQwater) by adding 2 mL of MilliQ water into 8 g of phenol.
Add 2 mL of each sample into large test tube.
To each standard and sample, add 0.05 mL of the phenol reagent.
Mix on a vortex.
To each standard and sample, add 5 mL of concentrated sulfuric acid. The sulfuric acid reagent should be added rapidly to the test tube. Direct the stream of acid against the liquid surface rather than against the side of the test tube in order to obtain good mixing. (These reactions are driven by the heat produced upon the addition of H₂SO₄ to an aqueous sample. Thus, the rate of addition of sulfuric acid must be standardized.)
Mix on a Vortex.
Let tubes stand for 10 min and then place in a 25° C. bath for 10 min (i.e. to cool them to room temperature).
Vortex the test tubes again before reading the absorbance.
Pour each standard and sample into a quartz corvette and use the standard of 0 g/L as blank.
Read the absorbance at 490 nm.
Discard the waste into glass waste bottle.
Total carbohydrate concentration can be calculated using the equation obtained from the standard curve.
Suspend 0.1 g of freeze-dry biomass into 1 mL of MilliQ water (in triplicates)
Actual weight: a. 0.1021 g
   b. 0.0999 g
   c. 0.1005 g
Make 1 in 1000 dilution for each sample in total volume 1 mL use MilliQ water
Vortex each sample vigorously
Transfer 0.4 mL of each sample into a large glass test tube with lid
Transfer 0.4 mL of MilliQ water into a large glass test tube with lid as blank
Add 0.01 mL of phenol reagent (80%) to each sample
Vortex each sample vigorously
Add 1 mL of concentrated sulphuric acid to each sample
Stand all samples at RT for 10 mins
Incubate all samples at 25° C. for 10 mins
Pour each blank and sample into a quartz corvette and read the OD at 490 nm

The total carbohydrate concentration can be calculated using the equation obtained from the standard curve.

Results

The total carbohydrate content of the algal biomass was determined to be 21.39%±1.67.

### Example 11

### Total Protein Content Analysis of the Algal Biomass

Materials

Freeze dried algal biomass was prepared according to Example 6.

Procedures

Samples were sent to NCRIS to do the total protein content analysis use TKN analyser.

Results

The total protein content of the algal biomass was determined to be 30.10%±1.66.

### Example 12

### Total Ash Content Analysis of the Algal Biomass

Materials

Freeze dried algal biomass was prepared according to Example 6.

Procedures

Samples were sent to NCRIS to do the total ash content analysis use.

Results

The total ash content of the algal biomass was determined as 16.96%±0.27.

Summary

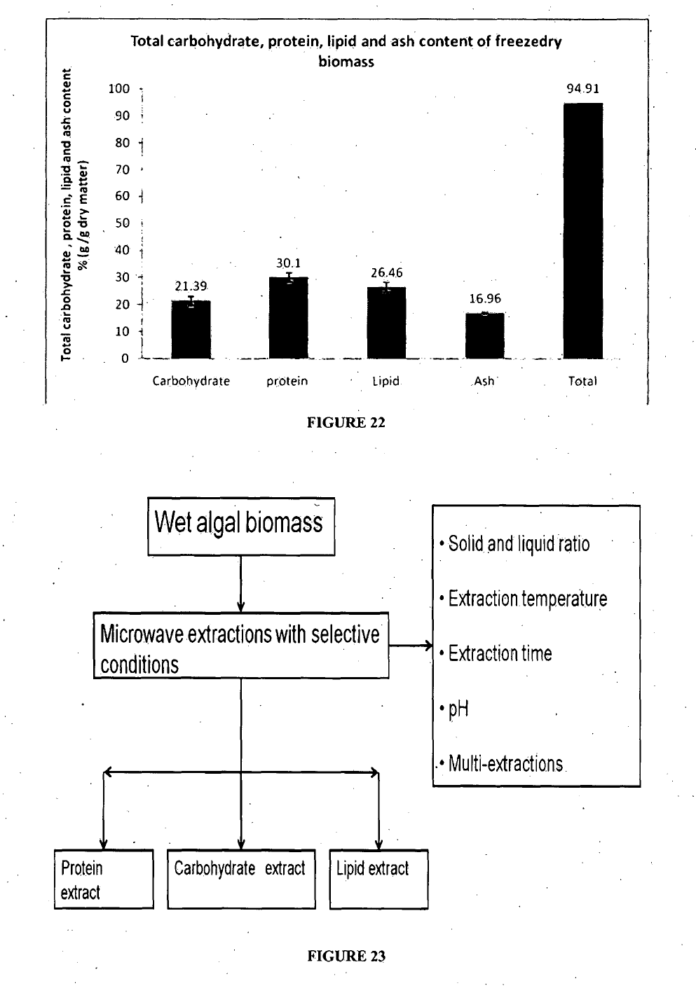

Total carbohydrate, protein and lipid content of the algal biomass was completed using three independent analytical methods, namely the phenol and sulphuric acid method, TKN method and Bligh and Dyer method, respectively. Furthermore, by combining the results of the ash content, the total dry content recovery is almost 95% (FIG. 22). With such a small difference, it is able to conclude that the chemical characterization of the algal biomass was completed and the accuracy of the results is trustable. Meanwhile, the current algal biomass primary metabolites profile will directly be used as the baseline of extraction efficiency to optimise the novel microwave solvent-free extraction technique.

### Example 13 Optimisation of the Microwave Solvent-Free Extraction Conditions with Wet Algal Biomass

The experimental design is shown in FIG. 23.

According to previous results, the batch of wet algal biomass being used contained 74% moisture and 26% dry content. Therefore, the solid and liquid ratio of the wet biomass was about 1:3.5. However, after some test runs of the microwave extractions using the wet biomass directly, the high solid content reduced the mixing efficiency of the extraction process as well as the energy transformation. Meanwhile, it also caused some difficulty in harvesting the product and re-starting the subsequent extraction. In order to solve these technical difficulties and standardize a solid and liquid ratio for further process development, the biomass was diluted to a 1:10 solid to liquid ratio. Compared to the original wet biomass, the diluted biomass was much easier to mix and transfer.

### Example 14

### Microwave Heating Time Optimisation

Materials

Frozen wet algal biomass was received from SARDI on Jun. 6, 2011.

Equipment

The microwave extraction unit used was a Milestone, Start Synth microwave synthesis labstation.

Procedures
Accurately weigh 7×10 g of wet biomass and place it into a 50 mL flat bottom flask.
Actual weight of each sample: a. 2-1 (2 mins heating with pH 1): 9.9565 g
   b. 10-1 (10 mins heating with pH 1): 9.9564 g
   c. 20-1 (20 mins heating with pH 1): 10.0357 g
   d. 30-1 (30 mins heating with pH 1): 10.0845 g
   e. 2-13 (2 mins heating with pH 13): 9.9564 g
   f. 10-13 (10 mins heating with pH 13): 10.0357 g
   g. 20-13 (20 mins heating with pH 13): 10.0845 g
Add 18.6 mL of 0.5 M H₂SO₄ solution to sample, 2-1, 10-1, 20-1 and 30-1 and mix well (the solid and liquid ratio will be 1:10).
Adjust the pH of each sample to 1 use 10 M KOH solution.
Add 18.6 mL of 0.5 M NaOH solution to sample, 2-13, 10-13 and 20-13 and mix well (the solid and liquid ratio will be 1:10).
Adjust the pH of each sample to 13 use 5 M H₂SO₄ solution.
Process the sample in microwave under the following conditions:
   Each sample is irradiated at microwave power of 1000 Watt until the sample temperature rises to 100° C.
   Keep irradiating each sample at 100° C. for 2, 10, 20, 30 mins with 200 W microwave power input.
After the process, cool the sample in water bath for 3 to 5 mins.
Transfer the sample into a centrifuge tube and centrifuge at 8,000 g for 10 mins.
Pour the supernatant into a 25 mL volumetric cylinder to measure the volume.
Supernatant volume: a. 2-1 (2 mins heating with pH 1): 17.5 mL
   b. 10-1 (10 mins heating with pH 1): 17.0 mL
   c. 20-1 (20 mins heating with pH 1): 20.0 mL
   d. 30-1 (30 mins heating with pH 1): 19.0 mL
   e. 2-13 (2 mins heating with pH 13): 17.5 mL
   f. 10-13 (10 mins heating with pH 13): 19.0 mL
   g. 20-13 (20 mins heating with pH 13): 19.0 mL
Re-suspend each pellet of sample 2-1, 10-1, 20-1 and 30-1 into 0.5 M NaOH solution according to the volume of supernatant been removed.
Adjust the pH of each sample to 13 using 5 M H₂SO₄ solution.
Re-suspend each pellet of sample 2-13, 10-13, 20-13 into 0.5 M H₂SO₄ solution according to the volume of supernatant been removed.
Adjust the pH of each sample to 1 use 10 M KOH solution.
Process the sample in microwave under the following conditions as the second extraction:
   Each sample is irradiated at microwave power of 1000 Watt until the sample temperature rises to 100° C.
   Keep heating the sample at 100° C. for 2, 10, 20, 30 mins with 200 W microwave power input.
After the process, cool the sample in water bath for 3 to 5 mins.
Transfer the sample into a centrifuge tube and centrifuge at 8,000 g for 10 mins.
Pour the supernatant into a 25 mL volumetric cylinder to measure the volume.
Supernatant volume: a. 2-1-13 (2 mins heating with pH 13): 17.5 mL
   b. 10-1-13 (10 mins heating with pH 13): 17.0 mL
   c. 20-1-13 (20 mins heating with pH 13): 20.0 mL
   d. 30-1-13 (30 mins heating with pH 13): 19.0 mL
   e. 2-13-1 (2 mins heating with pH 1): 17.5 mL
   f. 10-13-1 (10 mins heating with pH 1): 19.0 mL
   g. 20-13-1 (20 mins heating with pH 1): 19.0 mL
Store the pellet of each sample at −20° C. for further analysis
Analyse the total carbohydrate, protein and reducing sugar extraction efficiency of each extract.

### Example 14.1

### Protein Extraction Efficiency Analysis for the Microwave Extracts Obtained from Example 14 Using the Micro-Lowry Assay

Materials

The microwave extracts were samples 2-1, 10-1, 20-1, 30-1, 2-1-13, 10-1-13, 20-1-13, 2-13, 10-13, 20-13, 2-13-1, 10-13-1 and 20-13-1 from Example 14.

A total protein kit, micro-lowry, peterson's modification, Sigma codes: TP 0300 and L 3540 was used.

Procedures
Prepare a standard curve:
   Diluting the 0.4 mg/mL of the BSA protein standard solution in water in 1.5 mL centrifuge tubes according to the table below (in triplicates):

| Protein standard | Water | Protein |
|---|---|---|
| solution (mL) | (mL) | concentration (mg/mL) |
| 0.05 | 0.35 | 0.05 |
| 0.1 | 0.3 | 0.1 |
| 0.2 | 0.2 | 0.2 |
| 0.3 | 0.1 | 0.3 |
| 0.4 | 0 | 0.4 |

Use 0.4 mL of MilliQ water as the blank
Add 0.4 mL of the lowry reagent solution to the standard and blank.
Allow solutions to stand at room temperature for 20 mins.
With rapid and immediate mixing, add 0.2 mL of the folin & ciocalteu's phenol reagent working solution to each tube.
Allow colour to develop for 30 mins.
Transfer 0.3 mL of each standard and blank to a 96-well plate and read the absorbance at 620 nm.
Make 1 in 100 for each sample obtained from microwave extraction process in experiment 4 into the total volume 0.4 mL in a 1.5 mL centrifuge tube.
   For 1, in 10 dilutions: 0.1 mL of the sample+0.9 mL of MilliQ water
   For 1 in 100 dilutions: 0.04 mL of 10 time diluted sample+0.36 mL of MilliQ water
Use 0.4 mL of MilliQ water as the blank
Add 0.4 mL of the lowry reagent solution to the standard and blank.
Allow solutions to stand at room temperature for 20 mins.
With rapid and immediate mixing, add 0.2 mL of the folin & ciocalteu's phenol reagent working solution to each tube.
Allow colour to develop for 30 mins.
Transfer 0.3 mL of each standard and blank to a 96-well plate and read the absorbance at 620 nm.
According to the standard, the total protein concentration of each extract can be calculated; the total amount of reducing been collected can be calculated; the protein extraction efficiency can be calculated.

Results

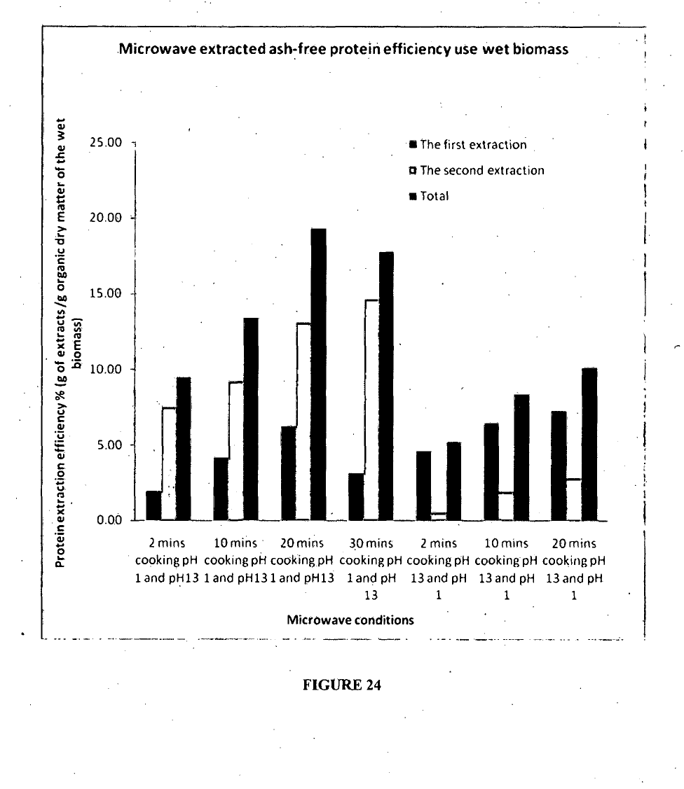

The results are shown in FIG. 24. The results show that, with alkaline condition as the first extraction, the total ash-free extraction efficiency was much lower than the total extraction efficiency with acidic condition as the first extraction. The 20 minute extraction under acidic conditions as the first extraction resulted in recovery of about 18% of the organic matter of the algal biomass in total. However, the 20 minute extraction under alkaline conditions as the first extraction only recovered about 10% of the organic matter of the algal biomass in total.

As shown in FIG. 24, the ash-free protein extraction efficiency clearly increased with longer extraction time from 2 minutes to 20 minutes, with not only the efficiency of the first extraction, but also the efficiency of the second extraction increasing. However, with even longer extraction time, the ash-free protein extraction efficiency decreased slightly, because the first acidic extraction recovered much less protein than other extractions with shorter extraction time. With such a long heating time, very low pH condition and high extraction temperature, the proteins might be damaged and unable to be detected by protein assay.

### Example 14.2

### Reducing Sugar Extraction Efficiency Analysis for the Microwave Extracts Obtained from Example 14 Using DNS Assay

Materials

The microwave extracts were samples 2-1, 10-1, 20-1, 30-1, 2-1-13, 10-1-13, 20-1-13, 2-13, 10-13, 20-13, 2-13-1, 10-13-1 and 20-13-1 from Example 14.

Procedures
Prepare a standard curve
   Dilute 1 mg/mL of glucose stock solution in water in 1.5 mL centrifuge tubes according to the table below (in triplicates):

| Protein standard | Water | glucose |
|---|---|---|
| solution (mL) | (mL) | concentration (mg/mL ) |
| 0.0 | 1.0 | 0.0 |
| 0.2 | 0.8 | 0.2 |
| 0.4 | 0.6 | 0.4 |
| 0.6 | 0.4 | 0.6 |
| 0.8 | 0.2 | 0.8 |
| 1.0 | 0.0 | 1.0 |

Use MilliQ water as the blank.
Transfer 0.1 mL of each standard solution and blank into 1.5 mL centrifuge tube.
Heat up all standards and blank to 90° C.
At 30 secs interval, add 0.05 mL of 0.5 M KOH solution and 0.1 mL of DNS reagent.
Incubate all standards and blank at 90° C. for 5 mins.
Cool all standards and blank on ice for 20 mins.
Add 0.75 mL of MilliQ water to each standard and blank.
Read the absorbance at 520 nm.
Make 1 in 10 for sample 2-1, 10-1, 20-1, 30-1, 2-1-13, 10-1-13, 20-1-13, 2-13, 2-13-1, 10-13-1 and 20-13-1.
   For 1 in 10 dilutions: 0.1 mL of the sample+0.9 mL of MilliQ water
Make no dilution for sample 10-13, 20-13.
Use MilliQ water as the blank.
Transfer 0.1 mL of each standard solution and blank into 1.5 mL centrifuge tube.
Heat up all standards and blank to 90° C.
At 30 secs interval, add 0.05 mL of 0.5 M KOH solution and 0.1 mL of DNS reagent.
Incubate all standards and blank at 90° C. for 5 mins.
Cool all standards and blank on ice for 20 mins.
Add 0.75 mL of MilliQ water to each standard and blank.
Read the absorbance at 520 nm.
According to the standard, the total protein concentration of each extract can be calculated; the total amount of protein been collected can be calculated; the reducing sugar extraction efficiency can be calculated.

Results

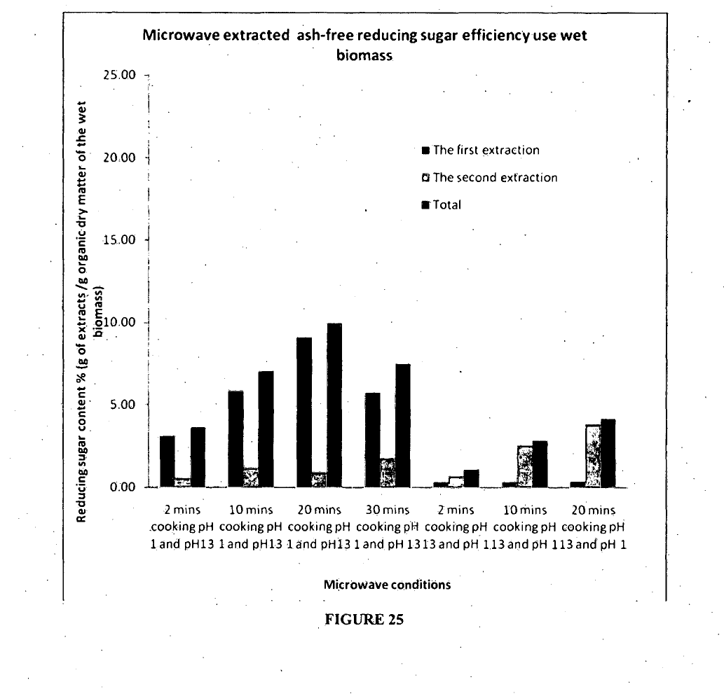

The results are shown in FIG. 25. Similar to the protein extraction, with alkaline conditions as the first extraction, the total ash-free extraction efficiency was much lower than the total extraction efficiency with acidic conditions as the first extraction. The 20 minute extraction with acidic conditions as the first extraction resulted in recovery about 11% of the organic matter of the algal biomass in total. However, the 20 minute extraction with alkaline conditions as the first extraction only recovered about 5% of organic matter of the algal biomass in total. As shown in FIG. 25, due to the low reducing sugar extraction efficiency under alkaline conditions, the total ash-free reducing sugar extracted was substantially contributed from the first acidic extraction. The ash-free reducing sugar extraction efficiency clearly increased with longer extraction time from 2 minutes to 20 minutes. However, with even longer extraction time, the ash-free reducing sugar extraction efficiency decreased slightly, because the first acidic extraction recovered much less reducing sugar than extraction with 20 minutes heating time.

### Example 14.3

### Total Carbohydrate Extraction Efficiency Analysis for the Microwave Extracts Obtained from Example 14 Using Phenol and Sulphuric Acid Assays

Materials

The microwave extracts were samples 2-1, 10-1, 20-1, 30-1, 2-1-13, 10-1-13, 20-1-13, 2-13, 10-13, 20-13, 2-13-1, 10-13-1 and 20-13-1 from Example 14.

Procedures

According to the method described in Example 14.2, the total carbohydrate concentration of each extract can be calculated; the total amount of carbohydrate collected can be calculated; the carbohydrate extraction efficiency can be calculated.

Results

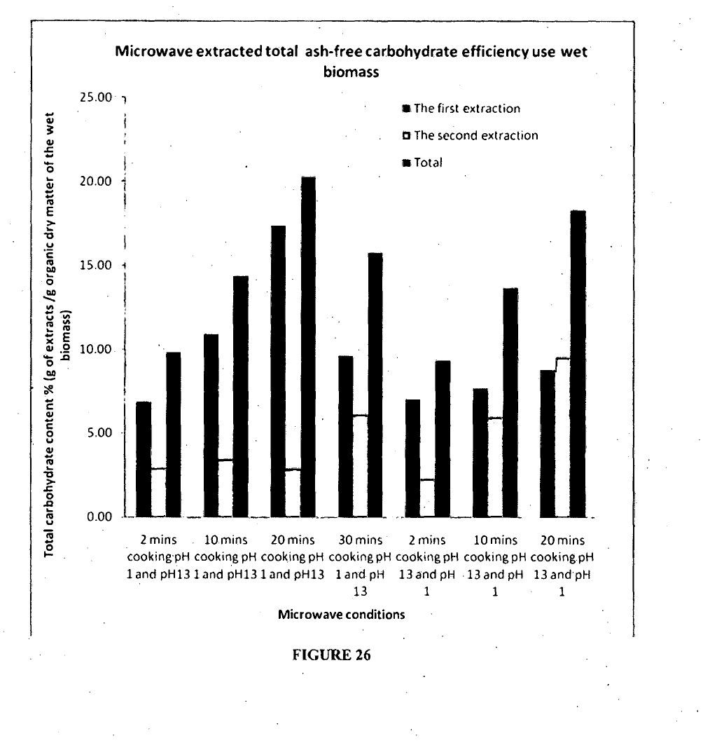

The results are shown in FIG. 26. Similar to the results from the protein and reducing sugar extractions, with alkaline condition as the first extraction, the total ash-free carbohydrate extraction efficiency was lower than the total extraction efficiency with acidic conditions as the first extraction. The 20 minute extraction with acidic conditions as the first extraction resulted in recovery of about 21% of the organic matter of the algal biomass in total. As shown in FIG. 26, due to the high carbohydrate extraction efficiency under acidic conditions, the total ash-free carbohydrate extracted was substantially contributed from the first acidic extraction as well. With alkaline conditions as the first extraction, some carbohydrate was extracted, but not as much as the acidic conditions. The ash-free carbohydrate extraction efficiency clearly increased with longer extraction time from 2 minutes to 20 minutes. However, with even longer extraction time, the ash-free carbohydrate extraction efficiency decreased, because the first acidic extraction recovered much less reducing sugar than extraction with 20 minutes heating time. The protein, reducing sugar and carbohydrate extraction efficiencies under different extraction times and pH conditions varied. However, as shown in FIGS. 24, 25 and 26, the 20 minute extraction with the first extraction under acidic conditions showed the highest protein, reducing sugar and carbohydrate extraction efficiencies. With longer extraction time, 30 minutes, less protein, reducing sugar and carbohydrate were extracted. With alkaline conditions as the first extraction and acidic conditions as the second extraction, beside the total carbohydrate, protein and reducing sugar extraction efficiency were much less. Therefore, it appears to be advantageous for the first extraction to be carried out under acidic conditions. This not only results in the recovery of more carbohydrate, but also helps to increase the protein extraction efficiency, presumably by removing the carbohydrate skeleton of the cell wall.

### Example 15

### Maximising the Ash-Free Protein and Carbohydrate Extraction Efficiency by Carrying Out a Third Extraction Using Leftover Material from the Second Extraction

Materials

The microwave extracts were samples 2-1-13, 10-1-13 and 20-1-13 from Example 14. Leftover material from the second microwave extraction was also used.

Procedures
Add certain volume of 0.5 M NaOH solution to the leftover material of sample, 2-1-13, 10-1-13 and 20-1-13 and mix well, according the volume of second extract.
Adjust the pH of each sample to 13 use 5 M H₂SO₄ solution.
Process the sample in microwave under the following conditions:
   Each sample is irradiated at microwave power of 1000 Watt until the sample temperature rises to 100° C.
   Keep heating each sample at 100° C. for 2, 10, 20 mins with 200 W microwave power input.
After the process, cools the sample in water bath for 3 to 5 mins.
Transfer the sample into a centrifuge tube and centrifuge at 8,000 g for 10 mins.
Pour the supernatant into a 25 mL volume metric cylinder to measure the volume.
Supernatant volume: a. 2-1-13-13 (2 mins heating with pH 13): 22.5 mL
   b. 10-1-13-13 (10 mins heating with pH 13): 19.0 mL
   c. 20-1-13-13 (20 mins heating with pH 13): 19.0 mL
Store the pellet of each sample at −20° C. for further analysis
Analyse the total carbohydrate and protein extraction efficiency of each extract.

Results

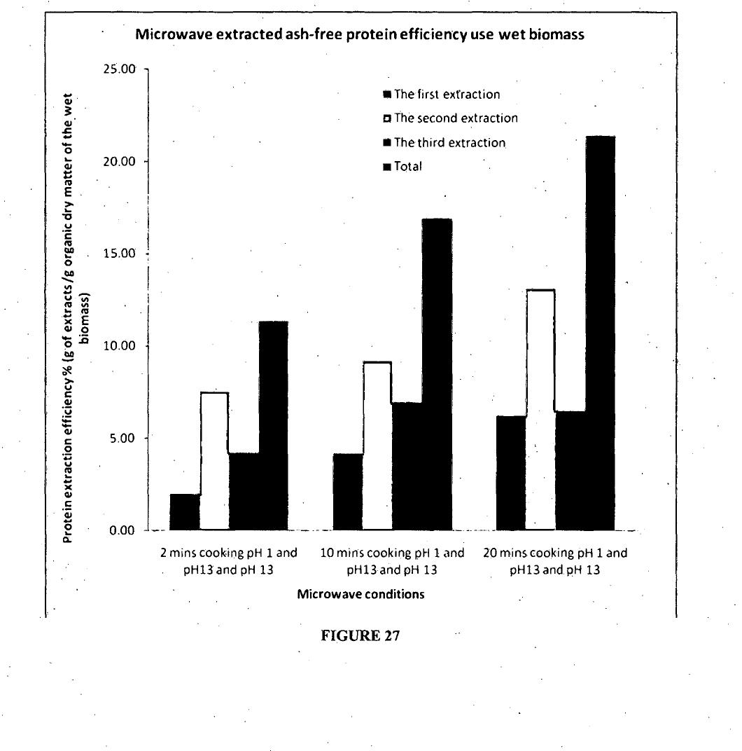

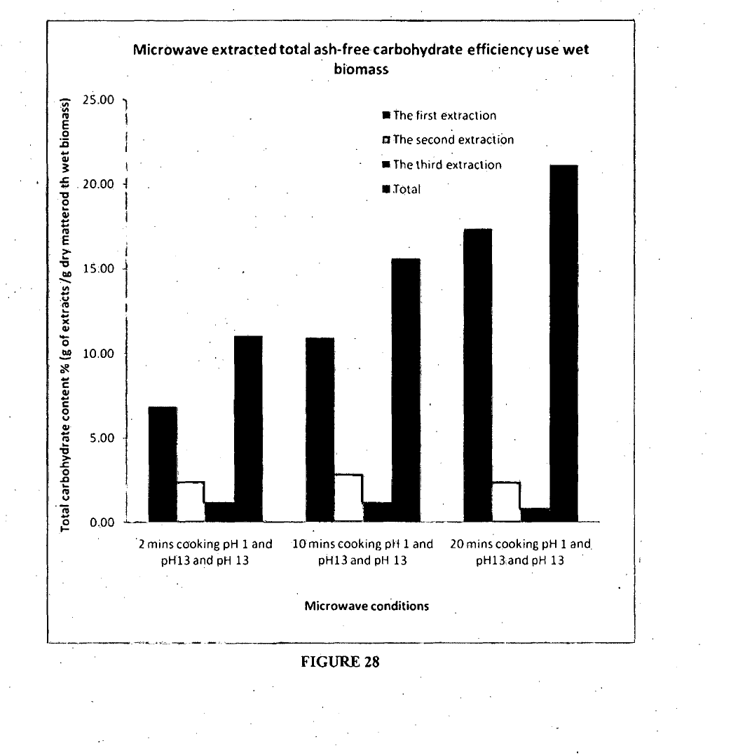

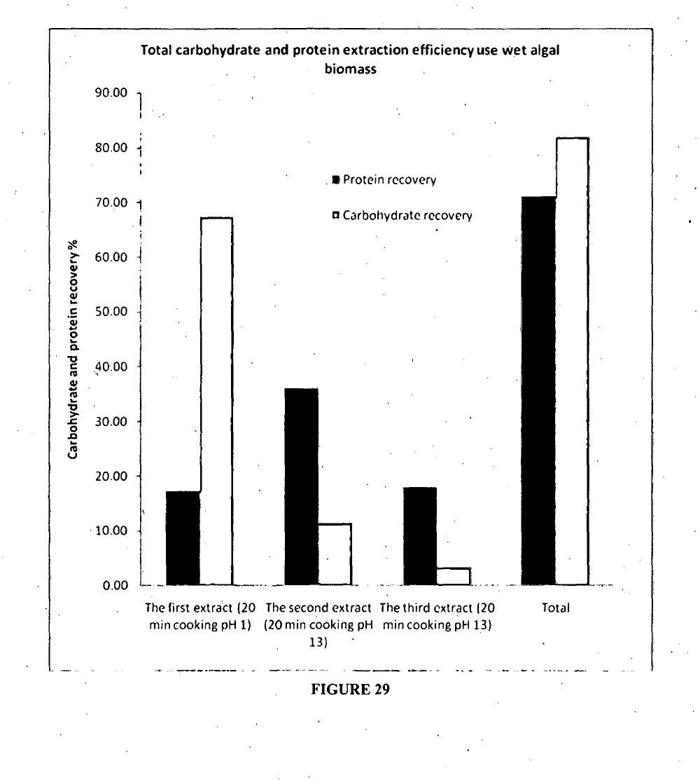

The results are shown in FIGS. 27, 28 and 29. With the third extraction, the total ash-free protein extraction efficiency increased from about 17% to about 21% of the organic matter of the biomass (FIG. 27). However, the total ash-free carbohydrate extraction efficiency did not increase too much. Furthermore, the total protein and carbohydrate recovery obtained from 20 mins extraction with acidic conditions followed by another two alkaline extractions (g of protein or carbohydrate recovered/g of actual protein or carbohydrate in the biomass) was high (FIG. 28). Through three extractions, about 70% of protein was collected and about 80% of carbohydrate was collected. Furthermore, the selectivity of each extraction was also good. The first acidic extract contained mainly carbohydrate and less much less protein; the second and third alkaline extracts contained mainly protein and less carbohydrate.

### Example 16

### Material Mass Balance Analysis of the Microwave Extraction Process and Remaining Lipid Analysis of the Microwave Extraction Process

Materials

Leftover material from the third microwave extraction from Example 15 was used.

Procedures
Freeze all leftover material, the pellet, of each sample use LN
Freeze dry all leftover materials use freeze-dryer for 48 hours
Weigh each dry pellet:
   P₂₋₁₋₁₃₋₁₃: 1.13 g
   P₁₀₋₁₋₁₃₋₁₃: 1.15 g
   P₂₀₋₁₋₁₃₋₁₃: 0.76 g
Take 0.1 g of each sample to analyse the lipid content use Bligh and Dyer method.

Results

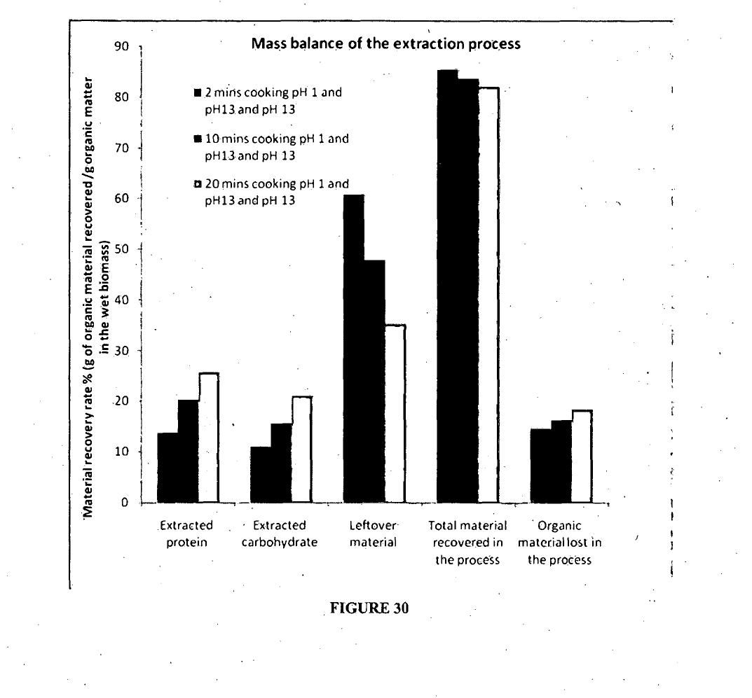

The results are shown in FIG. 30. As shown in FIG. 30, all multiple microwave extraction processes with different extraction times showed more than 80% material recovery rate. Only 15% to 20% of biomass was lost through the entire extraction process. With longer heating time and higher protein and carbohydrate extraction efficiency, the amount of leftover material was also reduced and finally the total material recovery rate of 2 mins, 10 mins and 20 mins extraction were very close to each other.

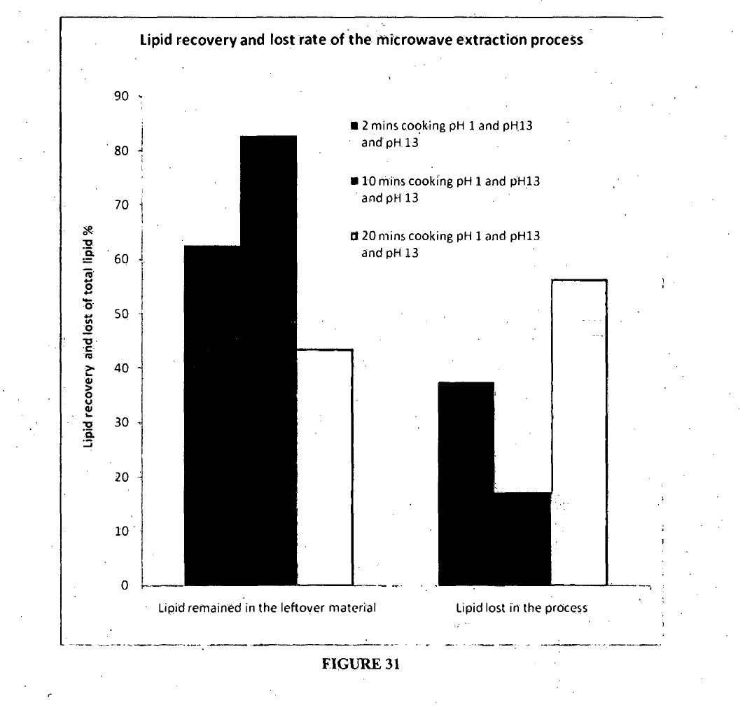

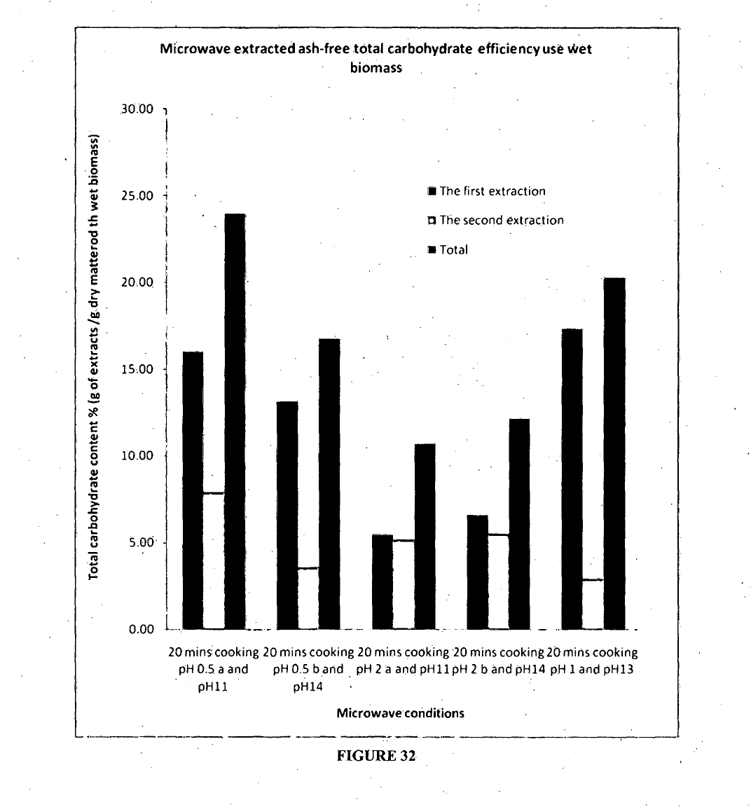

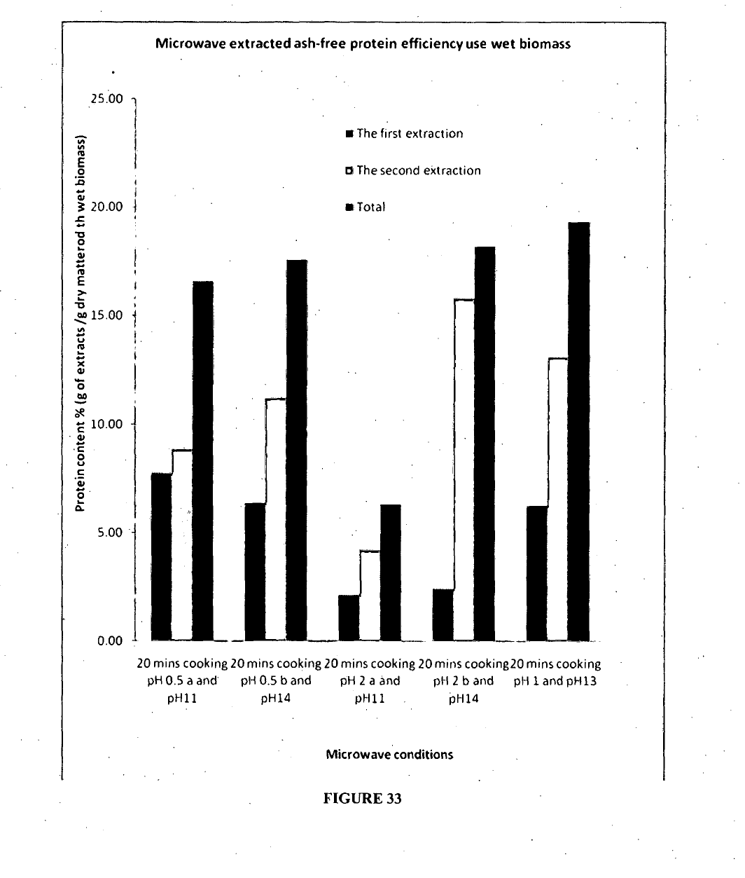

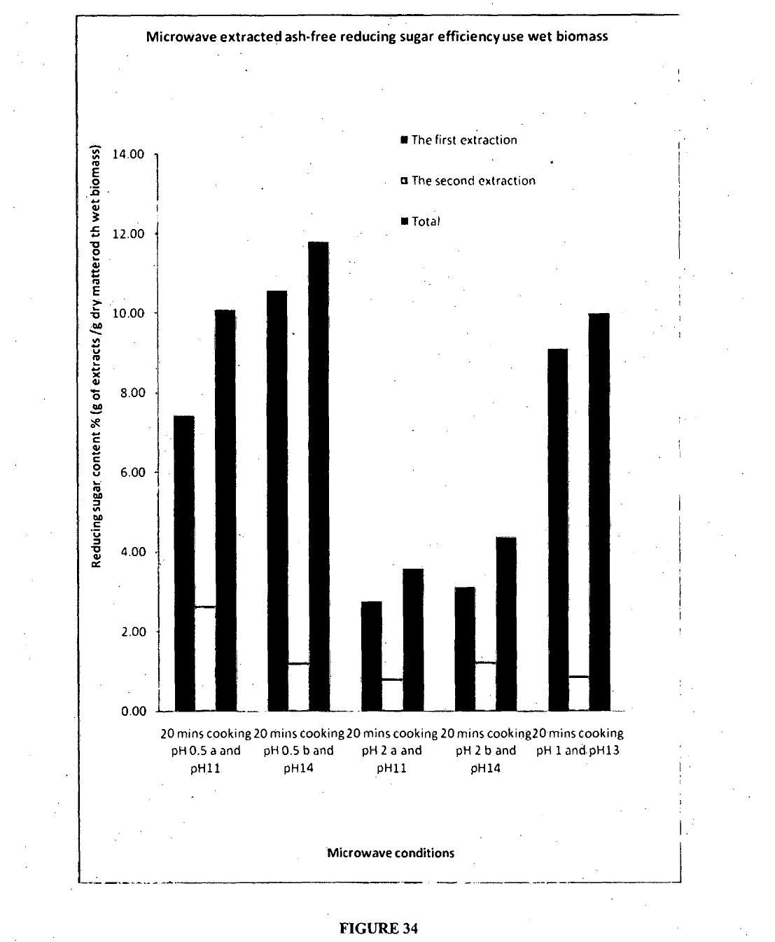

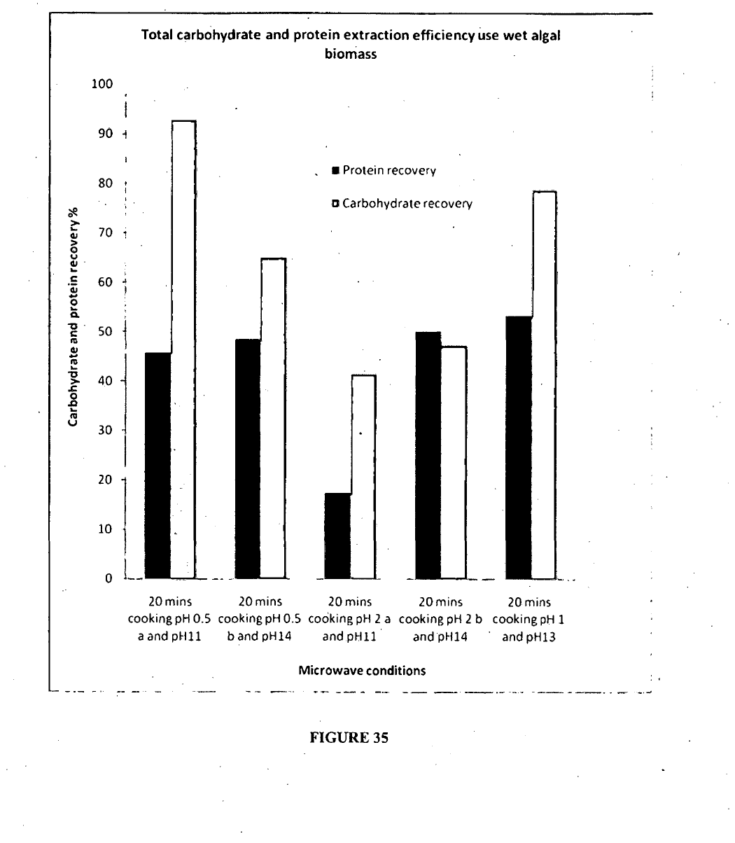

As a majority of the protein and carbohydrate had been removed after three microwave extractions, we expected to see some lipid released during the extraction process. However, it is difficult to measure the amount of lipid released into either the protein or the carbohydrate extracts. In order to determine how much lipid was lost during the process, the amount of lipid remaining within the leftover material was measured. As shown in FIG. 31, the maximum lipid recovery was about 83%, which was obtained from the extraction with 10 mins heating time. The lowest lipid recovery was about 43% which was obtained from the extraction with 20 mins heating time. With longer heating time and higher carbohydrate and protein extraction efficiency, the amount of lipid remaining should diminish. After carbohydrate has been removed, the lipid should no longer be locked inside the cell wall and, therefore, be more freely available for release to the extracts. However, the extraction process with 2 mins heating time showed fewer lipids remaining than with the extraction process with 10 mins heating time, but still more than the extraction process with 20 mins heating time.

### Example 17

### Microwave pH Conditions Optimisation with pH 0.5, 2, 11 and 14

Materials

Frozen wet algal biomass was received from SARDI on Jun. 6, 2011.

Procedures
Accurately weigh a 4×10 g of wet biomass and place it into a 50 mL flat bottom flask.
Actual weight of each sample: a. 0.5a (20 mins heating with pH 0.5): 10.0357 g
   b. 0.5b (20 mins heating with pH 0.5): 10.0427 g
   c. 2a (20 mins heating with pH 2): 9.8774 g
   d. 2b (20 mins heating with pH 2): 9.54475 g
Add 18.6 mL of 0.5 M H₂SO₄ solution to sample, 0.5a, 0.5b, 2a and 2b and mix well (the solid and liquid ratio will be 1:10).
Adjust the pH of sample 0.5a and 0.5 b to 0.5 using 5 M H₂SO₄ solution.
Adjust the pH of sample 2a and 2 b to 2 use 10 M KOH solution.
Process the sample in microwave under the following conditions:
   Each sample is irradiated at microwave power of 1000 Watt until the sample temperature rises to 100° C.
   Keep heating each sample at 100° C. for 2, 10, 20, 30 mins with 200 W microwave power input.
After the process, cool the sample in water bath for 3 to 5 mins.
Transfer the sample into a centrifuge tube and centrifuge at 8,000 g for 10 mins.
Pour the supernatant into a 25 mL volumetric cylinder to measure the volume.
Supernatant volume: a. 0.5a (20 mins heating with pH 0.5): 20 mL
   b. 0.5b (20 mins heating with pH 0.5): 17.5 mL
   c. 2a (20 mins heating with pH 2): 19 mL
   d. 2b (20 mins heating with pH 2): 19 mL
Re-suspend each pellet of sample 0.5 a, 0.5 b, 2 a and 2 b into 0.5 M NaOH solution according to the volume of supernatant been removed.
Adjust the pH of sample 0.5 a and 2 a to 11 using 5 M H₂SO₄ solution.
Adjust the pH of sample 0.5 b and 2 b to 14 using 10 M KOH solution.
Process the sample in microwave under the following conditions as the second extraction:
   Each sample is irradiated at microwave power of 1000 Watt until the sample temperature rises to 100° C.
   Keep heating the sample at 100° C. for 2, 10, 20, 30 mins with 200 W microwave power input.
After the process, cool the sample in water bath for 3 to 5 mins.
Transfer the sample into a centrifuge tube and centrifuge at 8,000 g for 10 mins.
Pour the supernatant into a 25 mL volumetric cylinder to measure the volume.
Supernatant volume: a. 0.5a-11 (20 mins heating with pH 11): 20 mL
   b. 0.5b-14 (20 mins heating with pH 14): 17.5 mL
   c. 2a-11 (20 mins heating with pH 11): 19 mL
   d. 2b-14 (20 mins heating with pH 14): 19 mL
Store the pellet of each sample at −20° C. for further analysis
Analyse the total carbohydrate, protein and reducing sugar extraction efficiency of each extract.

Results

To compare the pH condition 1 and 13 used in the previous experiment, lower and higher pH was investigated. Furthermore, in light of the previous results, 20 mins heating time was selected as the optimised heating time. Therefore, the extraction pH optimization is based on 20 mins heating time. For carbohydrate extraction, with lower pH, 0.5, the extraction efficiency was higher than the extraction efficiency with pH 2. However, it was also lower than the extraction efficiency with pH 1 in the previous study. The extractions at pH 0.5 and 14 gave the highest total carbohydrate extraction efficiency.

For protein extraction, extractions with pH 2 and 11 showed the lowest extraction efficiency. Furthermore, the extraction with pH 2 and 14 showed much higher extraction efficiency due to the second alkaline extraction at pH 14. However, the extraction with pH 1 and 13 in the previous study still showed the highest protein extraction efficiency, even compare to the extraction under much stronger conditions of pH 0.5 and 14.

For reducing sugar extraction, extractions with pH 2 and 11 and extractions with pH 2 and 14 showed the lowest extraction efficiency. This indicates that, beyond a certain pH, it is very difficult to extract reducing sugars. Furthermore, at pH 11 and 14, the amounts of reducing sugar extracted were low. The highest reducing sugar extraction efficiency was obtained from the extraction with pH 0.5 and 14.

With regards the recovery of carbohydrate and protein, the highest protein obtained so far is still from the extraction with pH 1 and 13 in the previous study. However, from the extraction with pH 0.5 and 11, the total carbohydrate recovery was higher than 90%.

### Example 18

### Comparison of Heating Methods

Materials

Frozen wet algal biomass was received from SARDI on Jun. 6, 2011.

Procedures

Samples were extracted at pH1 (first extraction), pH 13 (second extraction) and pH 13 (third extraction) using procedures of the earlier examples. However, three different heating methods were studied, namely heating on a hot plate for 20 minutes, heating in an autoclave for 20 minutes and heating in a microwave oven for 20 minutes.

The ash-free protein, ash-free carbohydrate and reducing sugar extraction efficiencies were then determined using the procedures set out earlier.

Results

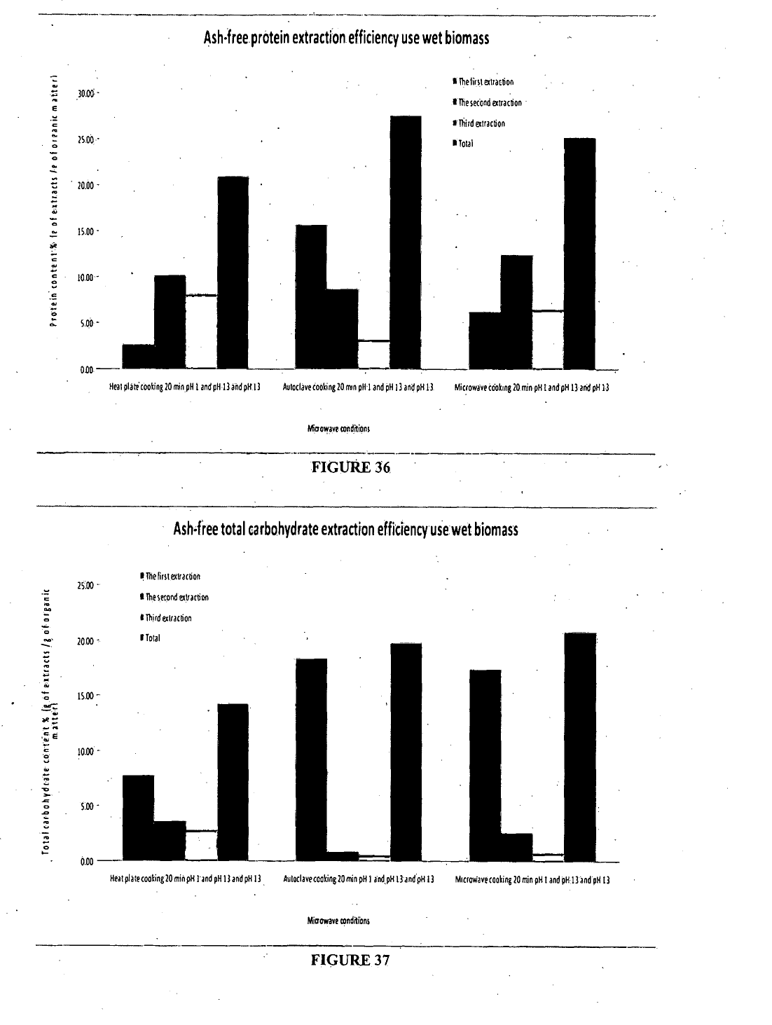

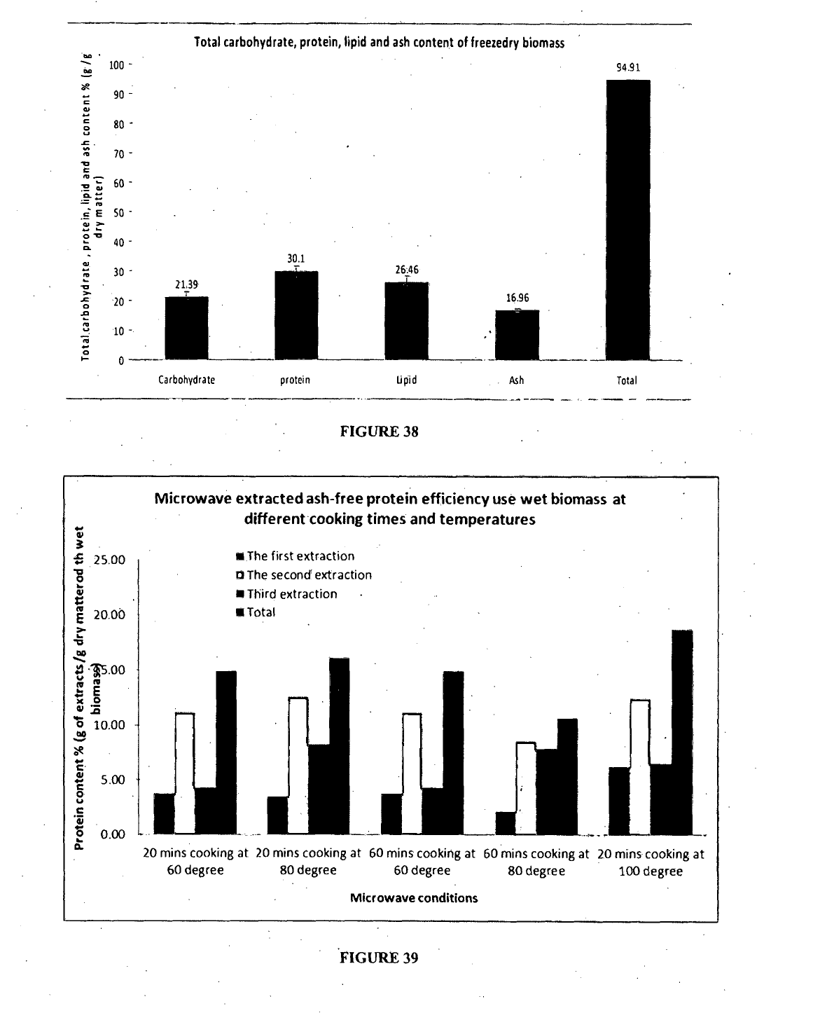

The results are shown in the Tables 5, 6 and 7 and in FIGS. 36, 37 and 38.

TABLE 5
Extraction efficiency for ash-free protein

| | Extractions | | | | Total ash- | Protein |
|---|---|---|---|---|---|---|
| | Extraction | Extraction | Extraction | Total | free protein | recovery |
| | 1 (mg) | 2 (mg) | 3 (mg) | (mg) | content (%) | (%) |
| Heat plate heating | 2.66 | 10.22 | 8.11 | 20.99 | 36.26 | 57.87 |
| 20 min pH 1 and | | | | | | |
| pH 13 and pH 13 | | | | | | |
| Autoclave heating | 15.68 | 8.66 | 3.23 | 27.57 | 36.26 | 76.03 |
| 20 min pH 1 and | | | | | | |
| pH 13 and pH 13 | | | | | | |
| Microwave | 6.23 | 12.44 | 6.49 | 25.15 | 36.26 | 69.36 |
| heating 20 min | | | | | | |
| pH 1 and pH 13 and | | | | | | |
| pH 13 | | | | | | |

TABLE 6
Extraction efficiency for ash-free total carbohydrate

| | Extractions | | | | Total ash-free | |
|---|---|---|---|---|---|---|
| | Extraction | Extraction | Extraction | Total | carbohydrate | Carbohydrate |
| | 1 (mg) | 2 (mg) | 3 (mg) | (mg) | content (%) | recovery (%) |
| Heat plate heating | 7.75 | 3.62 | 2.83 | 14.20 | 25.77 | 55.10 |
| 20 min pH 1 and | | | | | | |
| pH 13 and pH 13 | | | | | | |
| Autoclave heating | 18.30 | 0.88 | 0.56 | 19.74 | 25.77 | 76.60 |
| 20 min pH 1 and | | | | | | |
| pH 13 and pH 13 | | | | | | |
| Microwave | 17.35 | 2.56 | 0.82 | 20.73 | 25.77 | 80.44 |
| heating 20 min | | | | | | |
| pH 1 and pH 13 | | | | | | |
| and pH 13 | | | | | | |

TABLE 7
Extraction efficiency for reducing sugars

| Extractions | Extraction 1 (mg) |
|---|---|
| Heat plate heating 20 min pH 1 and pH 13 and pH 13 | 6.14 |
| Autoclave heating 20 min pH 1 and pH 13 and pH 13 | 18.63 |
| Microwave heating 20 min pH 1 and pH 13 and pH 13 | 9.09 |

In each case, heating on a hot plate resulted in less efficient extraction than heating in an autoclave or microwave oven.

### Example 19

### Optimisation of Microwave Temperature and Time Conditions with Lower Temperature and Longer Time

Materials

Frozen wet algal biomass was received from SARDI on Jun. 6, 2011.

Procedures
Accurately weigh a 4×10 g of wet biomass and place it into a 50 mL flat bottom flask.
Actual weight of each sample: a. 60a (20 mins heating at 60° C. with pH 1): 10.5987 g
   b. 60b (60 mins heating at 60° C. with pH 1): 10.1785 g
   c. 80a (20 mins heating at 80° C. with pH 1): 10.5544 g
   d. 80b (60 mins heating at 80° C. with pH 1): 10.2584 g
Add 18.6 mL of 0.5 M H₂SO₄ solution to sample, 60a, 60b, 80a and 80b and mix well (the solid and liquid ratio will be 1:10).
Adjust the pH of each sample 1 using 5 M H₂SO₄ solution.
Process the sample in microwave under the following conditions:
   Sample 60a is irradiated at microwave power of 1000 Watt until the sample temperature rises to 60° C. and keeps heating 20 mins with 200 W microwave power input.
   Sample 60b is irradiated at microwave power of 1000 Watt until the sample temperature rises to 60° C. and keeps heating 60 mins with 200 W microwave power input.
   Sample 80a is irradiated at microwave power of 1000 Watt until the sample temperature rises to 80° C. and keeps heating 20 mins with 200 W microwave power input.
   Sample 80a is irradiated at microwave power of 1000 Watt until the sample temperature rises to 80° C. and keeps heating 60 mins with 200 W microwave power input.
After the process, cool the sample in water bath for 3 to 5 mins.
Transfer the sample into a centrifuge tube and centrifuge at 8,000 g for 10 mins.
Pour the supernatant as the first extracts into a 25 mL volumetric cylinder to measure the volume.
   Supernatant volume: a. 60a (20 mins heating at 60° C. with pH 1): 20 mL
      b. 60b (60 mins heating at 60° C. with pH 1): 19 mL
      c. 80a (20 mins heating at 80° C. with pH 1): 20 mL
      d. 80b (60 mins heating at 80° C. with pH 1): 20 mL
Re-suspend each pellet of sample 60 a, 60b, 80a and 80b into 0.5 M NaOH solution according to the volume of supernatant removed.
Adjust the pH of each sample to 13 using 5 M H₂SO₄ solution.
Process the sample in microwave under the following conditions as the second extraction:
   Sample 60a is irradiated at microwave power of 1000 Watt until the sample temperature rises to 60° C. and keeps heating 20 mins with 200 W microwave power input.
   Sample 60b is irradiated at microwave power of 1000 Watt until the sample temperature rises to 60° C. and keeps heating 60 mins with 200 W microwave power input.
   Sample 80a is irradiated at microwave power of 1000 Watt until the sample temperature rises to 80° C. and keeps heating 20 mins with 200 W microwave power input.
   Sample 80a is irradiated at microwave power of 1000 Watt until the sample temperature rises to 80° C. and keeps heating 60 mins with 200 W microwave power input.
After the process, cool the sample in water bath for 3 to 5 mins.
Transfer the sample into a centrifuge tube and centrifuge at 8,000 g for 10 mins.
Pour the supernatant as the second extracts into a 25 mL volumetric cylinder to measure the volume.
Supernatant volume: a. 60a (20 mins heating at 60° C. with pH 13): 21 mL
   b. 60b (60 mins heating at 60° C. with pH 13): 19 mL
   c. 80a (20 mins heating at 80° C. with pH 13): 20 mL
   d. 80b (60 mins heating at 80° C. with pH 13): 21 mL
Re-suspend each pellet of sample 60a, 60b, 80a and 80b into 0.5 M NaOH solution according to the volume of supernatant removed.
Adjust the pH of each sample to 13 using 5 M H₂SO₄ solution.
Process the sample in microwave under the following conditions as the second extraction:
   Sample 60a is irradiated at microwave power of 1000 Watt until the sample temperature rises to 60° C. and keeps heating 20 mins with 200 W microwave power input.
   Sample 60b is irradiated at microwave power of 1000 Watt until the sample temperature rises to 60° C. and keeps heating 60 mins with 200 W microwave power input.
   Sample 80a is irradiated at microwave power of 1000 Watt until the sample temperature rises to 80° C. and keeps heating 20 mins with 200 W microwave power input.
   Sample 80a is irradiated at microwave power of 1000 Watt until the sample temperature rises to 80° C. and keeps heating 60 mins with 200 W microwave power input.
After the process, cool the sample in water bath for 3 to 5 mins.
Transfer the sample into a centrifuge tube and centrifuge at 8,000 g for 10 mins.
Pour the supernatant as the third extracts into a 25 mL volumetric cylinder to measure the volume.
Supernatant volume: a. 60a (20 mins heating at 60° C. with pH 13): 20 mL
   b. 60b (60 mins heating at 60° C. with pH 13): 19 mL
   c. 80a (20 mins heating at 80° C. with pH 13): 20 mL
   d. 80b (60 mins heating at 80° C. with pH 13): 20 mL
Store the pellet of each sample at −20° C. for further analysis
Analyse the total carbohydrate, protein and reducing sugar extraction efficiency of each extract.

Results

As shown in FIG. 39, the ash-free protein extraction at lower extraction temperatures, 60° C. and 80° C., with 20 minutes extraction time showed similar efficiencies, which were all around 15% of total weight of the biomass in total. Compared to the extraction at 100° C. for 20 minutes, the ash-free protein extraction efficiency at lower temperature was lower. Furthermore, with longer extraction time, 60 minutes, the ash-free protein extraction at lower extraction temperature, 60° C. still showed the similar extraction efficiency to the result obtained from 20 mins extraction at 60° C. The ash-free protein extraction at 80° C. with 60 minutes extraction time showed much lower efficiency than the result obtained from 20 mins extraction at 80° C. Therefore, by increasing the extraction time and lowering the extraction temperature, the ash-free protein extraction efficiency was not improved.

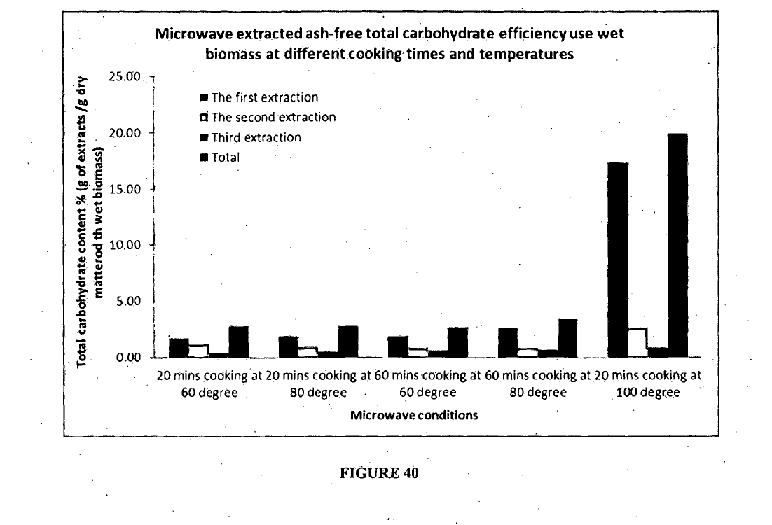

As shown in FIG. 40, the ash-free total carbohydrate extraction at lower extraction temperatures, 60° C. and 80° C., with 20 and 60 minutes extraction time showed similar efficiencies, which are all around 2% of total weight of the biomass in total. Compared to the extraction at 100° C. for 20 minutes, the ash-free protein extraction efficiency at lower temperature was much lower. It was clear that the first acidic extraction at lower temperature showed lower extraction efficiency. Therefore, by increasing the extraction time and lowering the extraction temperature, the ash-free total carbohydrate extraction efficiency was not improved.

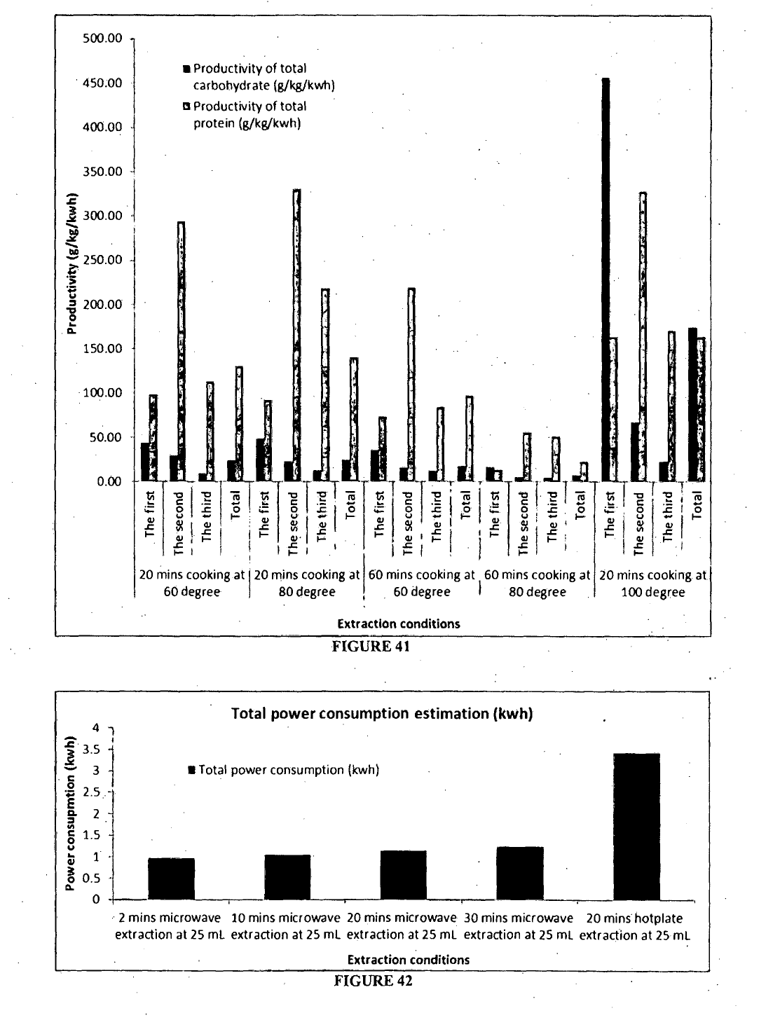

The productivity of protein and total carbohydrate extraction based on the power consumption at different extraction temperatures and times was calculated and the results are shown in FIG. 41. The productivity of total carbohydrate extraction based on the power consumption at lower extraction temperatures and longer extraction times was much lower than the optimised conditions, because of the low total carbohydrate extraction yield. Furthermore, productivities of protein extraction based on power consumption in total were still not improved by lowering the extraction temperature and increasing the extraction time.

### Example 20

### Power Consumption Estimation and Total Protein and Carbohydrate Productivity

The power consumption of a process as described herein using wet microalgae biomass, a microwave extraction unit, and extraction temperature of 100° C., an extraction time of 20 mins, a pH of 1 and 13, with three repeated extraction was estimated. Calculations showed that the major contribution of power consumption is the centrifuge, not the microwave. The power consumed during centrifugation takes about 90% of the total power consumption of the microwave extraction process. FIG. 42 shows that the total power consumption of microwave extractions for 2, 10, 20 and 30 mins were quite close to each other. However, the microwave heating process consumed much less power than the conventional hotplate heating process.

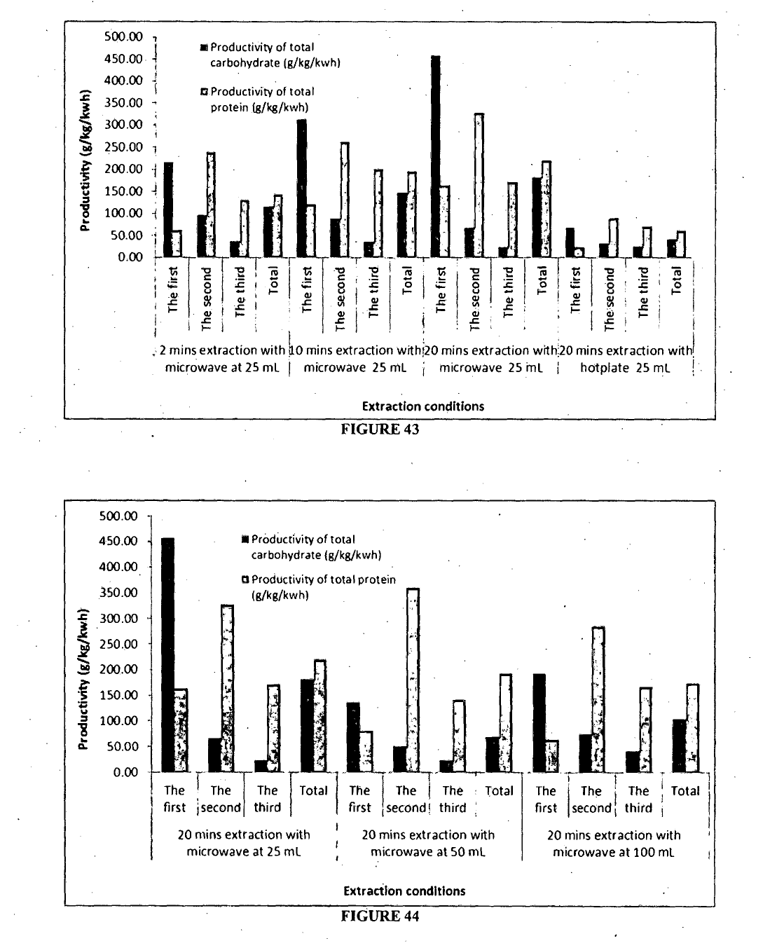

Furthermore, the productivity of total protein and carbohydrate based on the power consumption at different extraction conditions was calculated and the results are shown in FIGS. 43 and 44.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

1. A process for extracting lipid containing products from microalgal biomass, the process comprising: (i) treating an aqueous mixture comprising microalgal biomass with microwave radiation and (ii) recovering lipid containing products from the treated microalgal biomass.

2. The process according to claim 1, wherein the microalgal biomass is wet biomass comprising up to 90% water.

3. The process according to claim 2, wherein the microalgal biomass comprises 10% to 90% by weight of water.

4. A process for extracting lipid containing products and carbohydrate containing products from microalgal biomass, the process comprising: (i) providing an aqueous mixture containing the microalgal biomass; (ii) adjusting the pH of the aqueous mixture to pH<7; (iii) heating the aqueous mixture containing the microalgal biomass; and (iv) separating the lipid containing products and the carbohydrate containing products from the biomass.

5. A process for extracting lipid containing products and protein containing products from microalgal biomass, the process comprising: (i) providing an aqueous mixture containing the microalgal biomass; (ii) adjusting the pH of the aqueous mixture to pH>7; (iii) heating the aqueous mixture containing the microalgal biomass; and (iv) separating the lipid containing products and the protein containing products from the biomass.

6. A process for extracting lipid containing products, carbohydrate containing products, and protein containing products from microalgal biomass, the process comprising:
(i) providing an initial aqueous mixture containing the microalgal biomass;
(ii) adjusting the pH of the initial aqueous mixture to pH<7;
(iii) heating the acidic initial aqueous mixture containing the microalgal biomass to provide a first treated mixture;
(iv) separating the solid and the liquid from the first treated mixture to provide a first solid and a carbohydrate containing liquid;
(v) combining the first solid with an aqueous mixture to form a second aqueous mixture containing microalgal biomass;
(vi) adjusting the pH of the second aqueous mixture to pH>7;
(vii) heating the alkaline second aqueous mixture containing the microalgal biomass to provide a second treated mixture;
(viii) separating the solid and the liquid from the second treated mixture to provide a second solid and a protein containing liquid;
(ix) optionally, repeating steps (v) to (viii) using the second solid to provide a third solid and a further protein containing liquid;
(x) combining the second solid or the third solid with an aqueous mixture to form a final aqueous mixture;
(xi) treating the final aqueous mixture with a solvent;
(xii) Separating the solvent from the final aqueous mixture to provide a solvent containing lipid product.

7. The process according to claim 6, wherein the pH of the mixture formed in step (ii) is in the range of from 0.5 to 2.

8. The process according to claim 7, wherein the pH of the mixture formed in step (ii) is 1.

9. The process according to claim 6, wherein the pH of the mixture formed in step (vi) is in the range of from 11 to 14.

10. The process according to claim 9, wherein the pH of the mixture formed in step (iv) is 13.

11. The process according to claim 6, wherein the step of heating the acidic or alkaline aqueous mixture containing the microalgal biomass is carried out by treating the mixture with microwave radiation.

12. The process according to claim 11 wherein any one or more of the heating step(s) is carried out at atmospheric pressure.

13. The process according to claim 11 wherein any one or more of the heating step(s) is carried out at a pressure that is greater than atmospheric pressure.

14. A product produced by the process according to claim 1.

15. (canceled)
